(19) United States
(12) Patent Application Publication
(10) Pub. No.: US 2026/0207939 A1
(43) Pub. Date: Jul. 23, 2026

(54) SYSTEMS AND METHODS FOR TRANSCUTANEOUS AURICULAR VAGAL NERVE STIMULATION FOR CEREBRAL EDEMA ASSOCIATED WITH BRAIN INJURIES

(71) Applicant: Washington University, St. Louis, MO (US)

(72) Inventors: Eric Claude Leuthardt, St. Louis, MO (US); Anna Huguenard, St. Louis, MO (US); Rajat Dhar, St. Louis, MO (US)

(21) Appl. No.: 19/529,978

(22) Filed: Feb. 4, 2026

Related U.S. Application Data
(63) Continuation-in-part of application No. 18/548,755, filed on Sep. 1, 2023.
(60) Provisional application No. 63/156,792, filed on Mar. 4, 2021.

(51) Int. Cl. A61N 1/36; A61N 1/04

(52) U.S. Cl. CPC A61N 1/36034; A61N 1/0456; A61N 1/0472; A61N 1/36025; A61N 1/36036

(57) ABSTRACT

A method of reducing cerebral edema in a patient is provided. The method includes identifying the patient as having at least one of 1) having a condition associated with cerebral edema, or 2) being at risk for cerebral edema complications, positioning, on a skin of the patient, a neuromodulation device having an electrode, adjusting the neuromodulation device to position the electrode adjacent to at least one of a vagus nerve of the patient or a branch of a vagus nerve, and emitting an electrical signal at a predetermined intensity to stimulate the vagus nerve of the patient and to reduce cerebral edema by modulating cortical brain activity, thereby reducing metabolic burden on the brain, and activating anti-inflammatory pathways.

### CROSS REFERENCE TO RELATED APPLICATIONS

This application is a continuation in part of U.S. Patent Application No. 18/548,755, filed on September 1, 2023, which is a U.S. National Stage Entry of International Patent Application No. PCT/US2022/018864, filed March 4, 2022, which claims the benefit of and priority to U.S. Provisional Patent Application No. 63/156,792, filed March 4, 2021, which are hereby incorporated by reference in its entirety.

### BACKGROUND

The present disclosure generally relates to systems and methods of treatment of cerebral edema associated with brain hemorrhages.

Cerebral edema is a pathological condition characterized by abnormal accumulation of fluid within the brain parenchyma, leading to increased intracranial pressure, compromised cerebral perfusion, and potential brain herniation. Cerebral edema represents a common pathophysiological endpoint for numerous neurological conditions and constitutes a major contributor to morbidity and mortality in patients with acute brain injuries. The development of cerebral edema involves complex mechanisms including cytotoxic edema (cellular swelling due to ion pump failure), vasogenic edema (disruption of the blood-brain barrier leading to fluid extravasation), and interstitial edema (transependymal flow of cerebrospinal fluid). Inflammation and brain metabolism play a central role in the pathogenesis of cerebral edema across multiple etiologies, with inflammatory mediators directly contributing to blood-brain barrier disruption, cellular injury, and fluid accumulation.

In addition to inflammation, brain metabolic activity represents another important factor influencing the development and severity of cerebral edema. Increased cerebral metabolic activity elevates tissue oxygen demand and produces greater quantities of metabolic byproducts, which can exacerbate edema formation through several mechanisms. Heightened neuronal activity increases cerebral blood flow and capillary hydrostatic pressure, promoting fluid extravasation across the blood-brain barrier. Elevated metabolism also generates increased reactive oxygen species and other oxidative stress mediators that directly damage endothelial tight junctions and increase vascular permeability. Furthermore, increased metabolic activity amplifies excitotoxic injury through enhanced glutamate release, which contributes to cytotoxic edema via calcium-mediated cellular swelling. Conversely, reduction of brain metabolic activity has been demonstrated to attenuate cerebral edema formation. Therapeutic hypothermia, pharmacologically induced metabolic suppression, and sedation have all been shown to reduce cerebral metabolic rate and corresponding edema in experimental and clinical settings. The protective effect of reduced metabolism is mediated through decreased oxygen free radical production, attenuated inflammatory signaling, reduced excitotoxicity, and preservation of blood-brain barrier integrity.

Cerebral edema may manifest in distinct spatial and distributional patterns that carry important clinical and prognostic implications. Focal cerebral edema is localized to a discrete region of the brain, typically surrounding a primary lesion such as an intracerebral hemorrhage, tumor, or ischemic infarct. Focal edema contributes to mass effect, whereby the expanding edematous tissue exerts compressive forces on adjacent brain structures, resulting in distortion and displacement of normal anatomy. When sufficiently severe, mass effect produces midline shift, a lateral displacement of midline brain structures that serves as a critical radiographic indicator of impending neurological deterioration and herniation risk. In contrast, diffuse cerebral edema involves widespread fluid accumulation distributed throughout both cerebral hemispheres, resulting in global brain swelling characterized by generalized expansion of the brain parenchyma, effacement of sulci and cisterns, and bilateral compression of the ventricular system. Diffuse and global patterns of brain swelling are commonly observed in conditions such as traumatic brain injury, anoxic brain injury, fulminant hepatic failure, and severe systemic inflammatory states. Regardless of whether cerebral edema presents in a focal pattern with associated mass effect and midline shift or as diffuse global brain swelling, the resulting elevation in intracranial pressure compromises cerebral perfusion and drives secondary neurological injury, underscoring the critical need for effective therapeutic interventions that target the underlying inflammatory and pathophysiological mechanisms of edema formation.

Given the central role of inflammation in the pathogenesis of cerebral edema across these diverse etiologies, therapeutic approaches that modulate inflammatory responses hold significant promise for improving patient outcomes. The vagus nerve, through its anti-inflammatory reflex, represents a novel therapeutic target for attenuating neuroinflammation and cerebral edema. Vagal nerve stimulation has been shown to activate the cholinergic anti-inflammatory pathway, reducing the release of pro-inflammatory cytokines and preserving blood-brain barrier integrity. Accordingly, the systems and methods disclosed herein for vagal nerve stimulation may provide therapeutic benefit not only for patients with brain hemorrhages but also for patients suffering from cerebral edema secondary to ischemic stroke, traumatic brain injury, CNS infections, autoimmune disorders, and brain tumors.

Recent advances in neuroimmunology have established that the brain actively monitors and regulates peripheral immune responses through a concept termed “immunoception,” referring to the bidirectional functional loops between the brain and the immune system. The insular cortex (IC), recognized as the primary cortical site of interoception—the sensing of the body’s physiological state—has been shown to store immune-related information and to regulate peripheral inflammatory responses. The IC integrates interoceptive signals from the body, including those related to inflammation, via afferent pathways through the vagus nerve, the nucleus tractus solitarius (NTS), and the thalamus. This immune representation in the cortex allows the brain to generate physiologically synchronized responses to inflammatory conditions.

Scientific studies have demonstrated that neuronal ensembles in the IC that are activated during peripheral inflammatory conditions can be captured and subsequently reactivated to retrieve specific immune responses. In experimental models, chemogenetic reactivation of IC neuronal ensembles captured during colitis or peritonitis was sufficient to broadly retrieve the associated inflammatory state, even in the absence of the original inflammatory stimulus. This demonstrates that the brain can store and retrieve specific immune responses, extending the classical concept of immunological memory to include neuronal representations of inflammatory information stored in the cerebral cortex.

Anatomical studies have established that neurons in the IC that are active during peripheral inflammation project to autonomic nervous system control sites, including the dorsal motor nucleus of the vagus (DMV) and the rostral ventrolateral medulla (RVLM), which coordinate parasympathetic and sympathetic outflows, respectively. These anatomical connections enable the IC to influence peripheral immune activity through modulation of autonomic tone. Critically, inhibition of IC activity has been shown to attenuate peripheral inflammation. In experimental models of colitis, suppression of IC neuronal activity reduced clinical symptoms, decreased mucosal leukocyte infiltration, attenuated pro-inflammatory cytokine expression, and reduced activation of inflammatory cell populations including T cells and monocytes.

Intracerebral hemorrhages (ICH) account for 10-20% of all strokes and are associated with high rates of disability and mortality. Compression from hematoma contributes to the primary injury associate with ICH. Following an ICH, there is a significant risk of secondary brain injury caused by edema formation and blood breakdown. Second secondary injuries, such as, perihematomal edema (PHE) evolve in the first weeks following an ICH and can require acute medical intervention for treatment. An increase in edema can acutely lead to worsening midline shift and clinical determination.

Following an ICH, blood extravasation and heme breakdown can trigger an inflammatory cascade in a patient that in turn cases a systemic and local inflammatory response. This inflammation of neural tissue plays a central role in secondary brain injuries and PHE formation. Studies link serum cytokines with edema and health outcomes outcome. Activated microglia, activated neutrophil, and infiltrating leukocytes release pro-inflammatory cytokines. Particularly, elevated serum levels of interleukin-6 (IL-6), tumor necrosis factor-α (TNF-α), and matrix metalloproteinase-9 (MMP-9) have been associated with increased PHE volume and poorer functional outcomes in patients with ICH. For example, one study has linked an elevated serum level of IL-6 with a 30% increase in poor functional outcome in patients per 1 nanogram per liter of blood above baseline. Another study has linked IL-6 with a larger volume of PHE. These inflammatory mediators have been linked to blood-brain barrier disruption, which can lead to vasogenic edema and exacerbating secondary brain injury.

Another type of brain bleeds is subarachnoid hemorrhage (SAH). Between 3-5% of adults harbor an intracranial aneurysm, and 18-30% of those individuals have more than one aneurysm. Despite more frequent detection of unruptured aneurysms in the general population, many patients still present initially with a SAH. For patients presenting with SAH, the mortality rate is 10-25%, with an additional 30% of patients suffering permanent disability. Following SAH, there is a significant risk for an inflammatory response that causes early brain injury and edema, cerebral vasospasm, and delayed cortical ischemia that all contribute to the high morbidity to these patients.

The pathophysiology of aneurysm formation and rupture is complex and influenced by genetic and environmental factors. There is evidence that systemic and local inflammation may promote aneurysm formation and rupture and leads to poorer outcomes following SAH. T-cell and macrophage-mediated inflammation can mediate some histological changes within the vascular wall that leads to aneurysm formation, and macrophage infiltrates in the walls of ruptured aneurysms likely contribute to their fragility. Elevated levels of inflammatory mediators, complement, and vascular cell adhesion molecule-1 (VCAM-1) have also been demonstrated in aneurysms, compared to non-aneurysmal intracranial vessels. In aneurysms, cathepsin G, a serine protease produced primarily in neutrophils, can be found at the site of rupture, implicating neutrophils in the acute rupture process.

Following SAH, blood within the subarachnoid space triggers a local and systemic inflammatory response. Studies show that after SAH, there are increases in IL-1b, IL-6, IL-1, and TNF-α, within the CSF, increases in IL-1, IL-23, IL-17, and ICAM-1 in the serum, and increases in p-38 and p-MAPK in brain tissue. There is also evidence that inflammatory markers are correlated with patient outcomes. Elevated IL-6 has been associated with increased risk for vasospasm and poorer outcomes. Elevated IL-1b, IL-18, and TNF-α in the CSF are associated with cerebral edema and acute hydrocephalus. There is also evidence that the degree of leukocytosis alone on admission following SAH is associated with worse modified Rankin scale scores (mRS) on discharge.

Currently, there is no effective therapy to mitigate secondary injury following an ICH. The current approach for targeting inflammatory response following a brain injury (e.g., following an ICH, following an SAH, etc.) via pharmacologic interventions. While these pharmacologic interventions have mechanistic support for reducing inflammatory responses in patients, they have so far failed to reduce morbidity in brain injury patients. In smaller enrollment studies, there has been some promise of outcome improvement with Cyclosporin A and various types of steroids (methylprednisolone, hydrocortisone, and dexamethasone). Other medications demonstrated no impact on overall outcomes, like Clazosentan, Cilostazol, and IL-1 antagonists. In larger trials with >1000 patients, Simvastatin, Aspirin, non-steroidal anti-inflammatory medications, and thienopyridines all demonstrated no improvement in outcomes. Iron chelation and sphingosine 1-phosphate receptor analogs have also failed to show efficiency in reducing brain swelling during trials thereof. As such, pharmacologic approaches proven ineffective at mitigating secondary injuries following a brain injury (e.g., SAH, ICH, etc.) due to poor central nervous system penetrations, slow onset relative to the acute nature of brain injuries, and side effects of the medication. There are a number of additional neurological diagnoses that also are associated with significant cerebral edema.

Ischemic stroke represents a major cause of cerebral edema and affects approximately 795,000 individuals annually in the United States alone. Following arterial occlusion, the ischemic cascade initiates a series of events including excitotoxicity, oxidative stress, and inflammatory responses that culminate in cytotoxic and vasogenic edema. Malignant cerebral edema occurs in approximately 10% of ischemic stroke patients and is associated with mortality rates exceeding 80% without intervention. The inflammatory response following ischemic stroke involves activation of microglia, infiltration of peripheral immune cells, and release of pro-inflammatory cytokines including IL-1β, IL-6, and TNF-α, which exacerbate blood-brain barrier disruption and edema formation.

Traumatic brain injury (TBI) affects over 2.8 million individuals annually in the United States and is a leading cause of death and disability, particularly among young adults. Primary traumatic brain injury results from mechanical forces applied to the brain at the time of impact, causing contusions, axonal shearing, and vascular disruption. Secondary brain injury following TBI develops over hours to days and involves cerebral edema, ischemia, excitotoxicity, and neuroinflammation. Post-traumatic cerebral edema significantly contributes to elevated intracranial pressure and poor outcomes. The neuroinflammatory response following TBI involves activation of the innate immune system, complement activation, and cytokine release that perpetuate cellular injury and edema formation.

Central nervous system infections, including bacterial meningitis, viral encephalitis, and brain abscesses, represent another major cause of cerebral edema. Bacterial meningitis affects approximately 1.2 million individuals globally each year and is associated with mortality rates of 20-30% despite antibiotic treatment. The pathophysiology of meningitis-associated cerebral edema involves direct pathogen-mediated injury, release of bacterial toxins, and robust inflammatory responses including activation of the complement cascade, cytokine release, and recruitment of neutrophils and other immune cells. Viral encephalitis, caused by pathogens such as herpes simplex virus, arboviruses, and enterovirus, similarly induces cerebral edema through viral-mediated cellular injury and neuroinflammation. The inflammatory response in CNS infections contributes significantly to blood-brain barrier breakdown and edema formation.

Autoimmune and inflammatory disorders of the central nervous system, including multiple sclerosis, acute disseminated encephalomyelitis (ADEM), neuromyelitis optical spectrum disorder (NMOSD), autoimmune encephalitis, and CNS vasculitis, can cause cerebral edema through immune-mediated mechanisms. In these conditions, dysregulated immune responses directed against CNS antigens lead to inflammation, demyelination, and blood-brain barrier disruption. Autoimmune encephalitis, characterized by antibodies directed against neuronal surface antigens or synaptic proteins, can cause significant cerebral edema and life-threatening neurological complications. The inflammatory cascade in autoimmune CNS disorders involves both cellular and humoral immune responses that contribute to edema formation.

Brain tumors, both primary and metastatic, represent a significant cause of peritumoral cerebral edema. Peritumoral edema is predominantly vasogenic in nature and results from tumor-secreted factors that increase blood-brain barrier permeability, including vascular endothelial growth factor (VEGF), matrix metalloproteinases, and pro-inflammatory cytokines. The extent of peritumoral edema often exceeds the size of the tumor itself and contributes substantially to mass effect, neurological symptoms, and patient morbidity. High-grade gliomas, meningiomas, and brain metastases are particularly associated with significant peritumoral edema. Current management of tumor-associated edema relies primarily on corticosteroids, which carry significant side effects with prolonged use.

### BRIEF DESCRIPTION

A method of reducing cerebral edema in a patient is disclosed herein. The method includes identifying the patient as having at least one of having a condition associated with cerebral edema, or 2) being at risk for cerebral edema complications, positioning, on a skin of the patient, a neuromodulation device having an electrode, adjusting the neuromodulation device to position the electrode adjacent to at least one of a vagus nerve of the patient or a branch of a vagus nerve, and emitting an electrical signal at a predetermined intensity to stimulate the vagus nerve of the patient and to reduce cerebral edema by modulating cortical brain activity, thereby reducing metabolic burden on the brain, and activating anti-inflammatory pathways.

A system for treating cerebral edema in a patient is disclosed herein. The system includes an adjustable earpiece including a body, a first boss extending from the body, the first boss including a first electrode, and a second boss extending from the body, the second boss including a second electrode, the second boss moveable relative to the first boss, and an electrical stimulation device configured to stimulate a vagus nerve of the patient by applying an electrical stimulation via at least one of the first electrode or the second electrode, the electrical stimulation device electrically coupled to the adjustable earpiece.

An earpiece configured to apply an electrical signal to vagus nerve of a patient is disclosed herein. The earpiece includes a body having a first side and a second side, a lid coupled to the first side of the body, a first boss extending from the second side of the body, the first boss including a first electrode, and a second boss extending from the second side of the body, the second boss including a second electrode, the second boss being moveable relative to the first boss, and a power interface to electrical couple the first electrode and the second electrode to an electrical signal source.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 illustrates a system for providing vagal nerve stimulation to a patient in accordance with at least one embodiment.

FIG. 2 illustrates placement of electrodes on an ear of a patient for non-invasive transcutaneous vagus nerve stimulation using the system shown in FIG. 1.

FIG. 3A illustrates a perspective view of an earpiece including electrodes for non-invasive transcutaneous vagus nerve stimulation using the system shown in FIG. 1 and implemented in accordance with teachings of this disclosure.

FIG. 3B is a front view of a body of the earpiece of FIG. 3A.

FIG. 3C illustrates a perspective view of the earpiece of FIG. 3A in a contracted position.

FIG. 3D illustrates a perspective view of the earpiece of FIGS. 3A and 3B in an expanded position.

FIG. 3E illustrates a perspective view of the earpiece of FIGS. 3A-3C coupled to the patient’s ear.

FIG. 4 illustrates a process for providing vagal nerve stimulation using the system shown in FIG. 1.

FIG. 5 illustrates an example configuration of a client system shown in FIG. 1, in accordance with one embodiment of the present disclosure.

FIG. 6 illustrates the effect of transcutaneous auricular vagus nerve stimulation (taVNS) on cerebral edema in patients with subarachnoid hemorrhage (SAH).

FIGS. 7A and 7B illustrate the effect of transcutaneous auricular vagus nerve stimulation (taVNS) on perihematomal edema in patients with intracerebral hemorrhage (ICH).

FIG. 8 illustrates the amplitude-dependent effects of transcutaneous auricular vagus nerve stimulation (taVNS) on cortical high gamma activity recorded via intracranial electrodes in human subjects.

FIGS. 9A-9X illustrates various electrode positioning and adjustability mechanisms for accommodating anatomical variations between patients and ensuring optimal electrode contact with auricular target regions.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

The therapeutic implications of these findings are significant for the treatment of brain swelling and cerebral edema. The present disclosure provides a comprehensive multi-mechanism approach to reducing cerebral edema through transcutaneous auricular vagus nerve stimulation (taVNS) delivered via adjustable, personalizable electrodes configured to accommodate individual patient anatomy. This approach operates through three distinct but synergistic mechanisms: (1) reduction of systemic and central inflammation via the peripheral cholinergic anti-inflammatory pathway, wherein stimulation activates efferent vagal pathways that culminate in acetylcholine release and suppression of pro-inflammatory cytokine production in macrophages and microglia; (2) reduction of central brain activation and associated metabolic burden on the brain, wherein low-amplitude stimulation suppresses high gamma cortical activity across multiple brain regions, thereby reducing cerebral metabolic demand, oxygen consumption, and the metabolic byproducts that contribute to edema formation; and (3) reduction of activity specifically in the insular cortex, which serves as the primary cortical hub for immunoception and immune regulation, wherein attenuated insular activity interrupts the bidirectional amplification loops between the brain and peripheral immune system that can perpetuate and exacerbate inflammatory conditions. By attenuating cortical representations of inflammation stored in the insular cortex, vagal nerve stimulation may prevent the brain from maintaining and reinforcing peripheral inflammatory states. This triple mechanism—combining peripheral anti-inflammatory effects, central metabolic suppression, and targeted modulation of immune representations in the insular cortex—provides a comprehensive therapeutic approach to reducing cerebral edema in patients with brain hemorrhages and other acute neurological conditions.

Devices and methods of treatment for brain injuries such as subarachnoid hemorrhages (SAHs), intracerebral hemorrhages (ICHs) are disclosed. In some aspects, the method includes administering vagal nerve stimulation (VNS) to a patient in need. In some aspects, VNS may be administered using any suitable method including, but not limited to, cervical neck dissection and placement of a cuff electrode directly on the nerve, and non-invasive transcutaneous stimulation of the auricular branch of the vagus nerve. In other aspects, VNS can be administered transcutaneously through the neck to stimulate the nerve through the cervical region where the nerve resides in the carotid sheath. In some aspects, the transcutaneous stimulation of the auricular branch of the vagus nerve is implemented using a portable transcutaneous electrical nerve stimulation (TENS) unit connected to two ear clip electrodes positioned in an ear of the subject. In some aspects, the stimulation in the ear is contained in an ear-mounted system without wires or a separate TENS unit. In some such examples, the earpiece includes an integrated battery.

In another aspect, the portable TENS unit can apply taVNS via an earpiece with integrated electrodes. In some aspects, the earpiece includes an ear canal member that is configured to be disposed with the ear canal of a patient to secure the earpiece to the patient’s ear. In some aspects, the earpiece includes a first electrode configured to abut a cymba of the patient’s ear and a second electrode configured to abut a cavum of the patient’s ear. In some aspects, the relative position of the first electrode and the second electrode is adjustable to facilitate variance of the distance between the cavum and the cymba between patients. In some aspects, the second electrode is mounted on a rail of the earpiece to facilitate the movement thereof. In some aspects, the relative position of the ear canal member and the first electrode is adjustable to account for variance of the distance between the first electrode and the ear canal member between patients.

A method for treating a discrete or focal edema disclosed herein includes identifying the patient as having at least one of 1) having a brain hemorrhage or other condition associated with cerebral edema, or 2) being at risk for cerebral edema or brain swelling complications, positioning, in an ear of the patient, an adjustable earpiece having an electrode, adjusting the earpiece to position the electrode of the earpiece on a first portion of the patient’s ear, and emitting an electrical signal at a predetermined intensity to stimulate an auricular branch of the vagus nerve and thereby reduce cerebral edema through three synergistic mechanisms: (1) activating the peripheral cholinergic anti-inflammatory pathway to reduce systemic and central inflammation, (2) suppressing cortical brain activity to reduce metabolic burden on the brain, and (3) attenuating activity in the insular cortex to interrupt brain-immune amplification loops that perpetuate inflammatory states.

A system for treating a discrete or focal edema is disclosed herein. The system includes an adjustable earpiece including a body, a first boss extending from the body, the first boss including a first electrode, and a second boss extending from the body, the second boss including a second electrode, the second boss moveable relative to the first boss to accommodate variations in patient ear anatomy, and an electrical stimulation device configured to stimulate an auricular branch of the vagus nerve of the patient by applying an electrical stimulation via at least one of the first electrode or the second electrode, the electrical stimulation device electrically coupled to the adjustable earpiece and configured to deliver stimulation parameters selected to reduce cerebral edema through activation of anti-inflammatory pathways, suppression of cortical metabolic activity, and modulation of insular cortex activity.

Without being limited to any particular theory, the external ear is an effective position for non-invasive stimulation of the vagus nerve, where the auricular branch travels in the pinna of the ear. In some aspects, the ear clips and/or the earpiece used for the VNS treatment are positioned along the concha of the ear.

### AURICULAR ANATOMY AND NEURAL INNERVATION

The external ear (auricle or pinna) is a complex cartilaginous structure that collects and directs sound waves into the external auditory canal. The auricle comprises several distinct anatomical regions including the helix (the outer rim), antihelix (a Y-shaped prominence), tragus (the small projection anterior to the ear canal), antitragus (the prominence opposite the tragus), lobule (the earlobe), and the concha. The concha is the deepest concavity of the external ear and is divided into two distinct regions by the crus of the helix: the cymba conchae (the superior portion) and the cavum conchae (the inferior portion adjacent to the external auditory meatus).

The sensory innervation of the external ear is supplied by multiple cranial and cervical nerves. The auricular branch of the vagus nerve (ABVN), also known as Arnold’s nerve or the alderman’s nerve, provides sensory innervation to specific regions of the auricle. The ABVN arises from the superior ganglion of the vagus nerve, traverses the mastoid canaliculus, and emerges to innervate portions of the external ear. Critically, the cymba conchae and the cavum conchae receive the majority of their cutaneous innervation from the auricular branch of the vagus nerve, with studies demonstrating that approximately 100% of the cymba conchae and approximately 45% of the cavum conchae are innervated by the ABVN. The remaining portions of the external ear receive innervation from the great auricular nerve (C2-C3), the auriculotemporal branch of the trigeminal nerve, and the lesser occipital nerve.

The vagus nerve (cranial nerve X) is the longest cranial nerve and serves as the primary parasympathetic nerve of the body. The vagus nerve originates from the medulla oblongata and exits the skull through the jugular foramen alongside the internal jugular vein and internal carotid artery. The vagus nerve descends through the neck within the carotid sheath, positioned posteriorly between the internal jugular vein and the carotid artery. In the thorax, the vagus nerve provides innervation to the heart, lungs, and upper gastrointestinal tract. The auricular branch of the vagus nerve represents the only peripheral branch of the vagus nerve that is accessible for non-invasive transcutaneous stimulation, making the concha region of the ear an ideal target for neuromodulation therapy. Electrical stimulation of the cymba conchae and cavum conchae activates afferent vagal pathways that project to the nucleus tractus solitarius (NTS) in the brainstem, which in turn modulates autonomic and inflammatory responses throughout the body.

### THERAPEUTIC METHODS

Brain hemorrhages (i.e., brain bleeds, etc.), such as aneurysmal spontaneous subarachnoid hemorrhage (SAH) and intracerebral hemorrhage (ICH) are diseases with both high mortality and morbidity, with cerebral edema representing a major contributor to secondary brain injury and poor outcomes. Despite extensive research, few interventions have consistently demonstrated the ability to reduce cerebral edema and improve outcomes in these patients. Transcutaneous auricular vagus nerve stimulation (taVNS), delivered via adjustable electrodes that can be personalized to individual patient ear anatomy, provides a novel, non-pharmacologic, non-invasive approach to reducing cerebral edema through three distinct mechanisms: (1) activation of the peripheral cholinergic anti-inflammatory pathway to reduce systemic and central inflammation, (2) suppression of cortical brain activity to reduce metabolic burden on the injured brain, and (3) targeted reduction of insular cortex activity to interrupt the bidirectional brain-immune amplification loops that perpetuate inflammatory states and edema formation. Vagal nerve stimulation (VNS) allows for more global regulation of the parasympathetic system rather than targeting a single inflammatory pathway like prior pharmaceutical approaches. Disclosed herein are systems and methods for reducing cerebral edema following a spontaneous subarachnoid hemorrhage (SAH), an intracerebral hemorrhage (ICH), and/or another brain hemorrhage or neurological condition associated with brain swelling, to improve clinical outcomes in those patients. The method disclosed herein includes administration of a therapeutically effective amount of vagal nerve stimulation (VNS) to treat, prevent, reduce, or reverse an inflammatory response following a spontaneous subarachnoid hemorrhage (SAH), an intracerebral hemorrhage (ICH), and/or another brain injury associated with harmful neuroinflammation (e.g., other TBIs, ischemic stroke, etc.

Vagus nerve stimulation has been shown to reduce inflammation. Substantial work has demonstrated that products of infection or injury activate sensory neurons traveling to the brainstem in the vagus nerve. The arrival of these incoming signals generates action potentials that travel from the brainstem to the spleen and other organs. This culminates in T cell release of acetylcholine, which interacts with α7 nicotinic acetylcholine receptors (α7 nAChR) on immunocompetent cells to inhibit cytokine release (e.g., release of IL-6, release of TNF-α, release of IL-1β, etc.) in macrophages and microglia. This neural-immunomodulatory circuit, referred to as the “cholinergic anti-inflammatory pathway. Additionally, acetylcholine facilitates the preservation of integrity of the blood-brain barrier (BBB). Accordingly, reduced activation of macrophages, neutrophil, and microglia attenuates breakdown of the BBB and vasogenic edema, which limits PHE expansion and the associated negative effects thereof.

In prior medical interventions, activation of the cholinergic anti-inflammatory pathway via VNS has been successfully implemented in models of inflammatory conditions like induced neuroinflammation, cerebral ischemia/reperfusion, rheumatoid arthritis, sepsis, and inflammatory bowel diseases or colitis. Harnessing its anti-inflammatory effects, VNS has been used in a mouse model of cerebral aneurysms and SAH. Prior studies have discovered that pre-treatment with VNS not only reduced the rupture rate of intracranial aneurysms, but also reduced the grade of hemorrhage if rupture occurred and improved survival and outcome after SAH.

The inventors have discovered applying VNS (e.g., taVNS, etc.) in the acute period following an ICH (e.g., a spontaneous ICH, etc.) attenuates the expected inflammatory response in a patient and will curtail ICH-induced perihematomal edema and the morbidity associated therewith. The inventors have discovered that applying taVNS to a patient following an ICH reduces inflammatory markers in patients following an ICH and the incidence of inflammation-mediated radiographic and biologic sequelae of ICH. The application of taVNS mitigates PHE by causing acetylcholine release and the engagement of cholinergic anti-inflammatory pathways. Particularly, as disclosed herein, taVNS applied after an ICH reduces inflammatory cytokines (e.g., TNF-α, etc.), reduces vasospasm, and improves neurologic outcomes in patients. Examples medical interventions disclosed herein offer significantly enhanced patient outcomes when compared to current pharmacologic approaches by reducing post-ICH morbidity.

As disclosed herein, VNS applied with certain characteristics can reduce the metabolic activity of particular brain regions associated with inflammatory responses. For example, VNS therapy applied in the sub-preceptive range (e.g., with an intensity of less than 0.5 milliamps (mA), etc.) can reduce high gamma brain wave activity in the insula (i.e., the insular cortex, etc.) and/or orbitofrontal cortex. The reduced neural activity can result in reduced metabolic activity in such brain regions, which reduces the inflammatory response and blood required by such regions. This reduction in inflammatory response and metabolic rate provides protection (e.g., neuroprotection, etc.) against secondary brain injury. As disclosed herein, combined with the inflammatory reduction associated with the activation of anti-inflammatory pathways, the reduction in neural activity associated with VNS in the sub-perceptive range substantially reduces the likelihood and severity of secondary brain injury following an SAH, an ICH, and/or another primary brain injury.

Historically, VNS was performed exclusively by surgical cervical neck dissection and placement of a cuff electrode directly around the nerve within the carotid sheath. In some aspects disclosed herein, VNS can be accomplished non-invasively by stimulating the auricular branch of the vagus nerve as it courses through the external ear, obviating the morbidity of a surgical procedure and allowing rapid deployment of the intervention in critically ill patients. The external ear is an ideal target for non-invasive stimulation of the vagus nerve, where the auricular branch travels in the concha of the ear.

Systems and methods disclosed herein are generally performed on a subject suffering from and/or at risk of a secondary brain injury following a brain hemorrhage, such as an SAH or an ICH. A subject in need of the therapeutic methods described herein can be a subject having, diagnosed with, suspected of having, or at risk for developing a spontaneous subarachnoid hemorrhage (SAH) and/or an intracerebral hemorrhage (ICH). In some embodiments disclosed herein, a determination of the need for treatment can be assessed by a history, physical exam, or diagnostic tests consistent with the disease or condition at issue. Diagnosis of the various conditions treatable by the methods described herein is within the skill of the art.

As used herein, a patient can refer to any subject receiving treatment for a brain hemorrhage, at risk for a brain hemorrhage, and/or a secondary injury related to a brain hemorrhage. In some embodiments disclosed herein, the subject is a human subject. However, it should be appreciated that, in other embodiments, the subject can be an animal subject, including a mammal, such as horses, cows, dogs, cats, sheep, pigs, mice, rats, monkeys, hamsters, guinea pigs, and humans or chickens.

Generally, a safe and effective intensity (e.g., current, amplitude, etc.) of vagal nerve stimulation (VNS) is, for example, an amount that would cause the desired therapeutic effect in a subject while minimizing undesired side effects (e.g., increased brain activity, pain to patient, discomfort to the patient, etc.). In various embodiments, an effective amount of vagal nerve stimulation (VNS) described herein can substantially inhibit an inflammatory response, slow the progress of an inflammatory response, or limit the development of an inflammatory response and/or a secondary brain injury associated with a spontaneous subarachnoid hemorrhage (SAH), an intracerebral hemorrhage (ICH), and/or another brain hemorrhage in a patient.

The primary goal of the non-invasive ear stimulation (auricular branch of the vagus nerve) delivered via adjustable, personalizable electrodes is to reduce cerebral edema following brain hemorrhages and other acute neurological conditions, thereby reducing the morbidity associated with these conditions. Reducing cerebral edema can in turn lead to reduction of hydrocephalus, reduction of vasospasm, reduction of secondary brain injury, reduction of intracranial pressure, reduction of infections, and/or reduction in ICU stay. Non-invasive ear stimulation can also be used to improve neurologic recovery from subarachnoid hemorrhage and intracerebral hemorrhage.

According to the methods described herein, the administration of VNS can be performed invasively or non-invasively. Non-limiting embodiments of suitable invasive methods for administering VNS include cervical neck dissection and placement of a cuff electrode directly on the vagus nerve. Non-limiting embodiments of suitable non-invasive methods for administering VNS include transcutaneous stimulation including, but not limited to, transcutaneous stimulation of the auricular branch of the vagus nerve using electrodes positioned on an ear of the subject. In some embodiments disclosed herein, the electrodes are incorporated into an adjustable reusable earpiece. In some embodiments disclosed herein, the electrodes are incorporated into a disposable earpiece. In some embodiments disclosed herein, the form factor of the stimulation devices can be in some embodiments disclosed herein, the adjustable earpiece can be mechanical changed to suit the anatomy of the ear of the patient receiving VNS. In some embodiments disclosed herein, neck form factors can be mechanical adjusted and/or deformed to accommodate the anatomy of the neck the patient receiving VNS.

The specific therapeutically effective dose level for any particular subject will depend upon a variety of factors including the disorder being treated and the severity of the disorder; the specific method of administration employed; the age, body weight, general health, sex, and diet of the subject; the time of administration; the route of administration; the duration of the treatment; drugs used in combination or coincidental with the specific method of administration employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503).

For example, it is well within the skill of the art to start doses of the composition at levels lower than those required to achieve the desired therapeutic effect and to gradually increase the dosage until the desired effect is achieved. If desired, the effective daily VNS dose may be divided into multiple doses for purposes of administration. Consequently, single dose VNS treatments may contain such amounts or submultiples thereof to make up the daily VNS dose. It will be understood, however, that the total daily usage of the VNS treatments of the present disclosure will be decided by an attending physician within the scope of sound medical judgment.

The systems and methods described herein are designed to benefit patients matching the states, diseases, disorders, and conditions described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

In some embodiments, administration of VNS can occur as a single event or over a time course of treatment. For example, VNS can be administered daily, weekly, bi-weekly, or monthly. For the treatment of acute conditions, the time course of treatment may be at least several days. Certain conditions could extend treatment from several days to several weeks. For example, treatment could extend over one week, two weeks, or three weeks. For more chronic conditions, treatment could extend from several weeks to several months or even a year or more. In some embodiments disclosed herein, a patient is treated with VNS at least two times within a twenty-four period.

Treatment in accord with the methods described herein can be performed prior to, concurrent with, or after conventional treatment modalities for brain hemorrhages (e.g., subarachnoid hemorrhages (SAH), intracerebral hemorrhages, etc.).

VNS can be administered simultaneously or sequentially with another agent, such as an antibiotic, an anti-inflammatory, or another agent. For example, VNS can be administered simultaneously with another agent, such as an antibiotic agent or an anti-inflammatory agent. Simultaneous administration can occur through the administration of VNS along with separate compositions, each containing one or more of an antibiotic agent, an anti-inflammatory agent, or another agent. Simultaneous administration can occur through the administration of VNS along with one composition containing two or more of agent(s), such as an antibiotic agent, an anti-inflammatory agent, or another agent. VNS can be administered sequentially with an antibiotic agent, an anti-inflammatory agent, or another agent. For example, VNS can be administered before or after administration of an antibiotic agent, an anti-inflammatory agent, or another agent.

To evaluate the effectiveness of vagal nerve stimulation (VNS) at reducing an inflammatory response associated with a spontaneous subarachnoid hemorrhage (SAH), the inventors conducted following experiments. Patients presenting with a spontaneous SAH were assigned to receive transcutaneous VNS using ear clip electrodes, or a sham treatment. Blood and CSF were collected from both groups and compared to assess the effect of VNS on various inflammatory biomarkers detected within the patient’s blood and CSF samples.

The results of these experiments demonstrated an attenuation of the inflammatory response in those patients associated with SAH treated using VNS as compared to the patients receiving sham treatments.

To evaluate the effectiveness of vagal nerve stimulation (VNS) at reducing an inflammatory response associated with intracerebral hemorrhages (ICH), the following experiments were conducted. Patients presenting with an intraparenchymal hemorrhage (IPH), a type of ICH, were assigned to receive transcutaneous VNS using an adjustable earpiece including integrated electrodes, or a sham treatment. Blood and CSF were collected from both groups and compared to assess the effect of VNS on various inflammatory biomarkers detected within the patient’s blood and CSF samples. The results of these experiments demonstrated an attenuation of the inflammatory response associated with ICH in those patients treated using VNS as compared to the patients receiving sham treatments.

In some embodiments, the electrical signal used to stimulate the auricular branch of the vagus nerve has an amplitude selected to produce suppression rather than activation of cortical activity. In such embodiments, the amplitude may be between 0.1 mA and 0.6 mA, or between 0.2 mA and 0.5 mA, or about 0.4 mA to 0.5 mA. In certain embodiments, the amplitude is selected to be at or below the subject’s perceptual threshold, such that the stimulation is not consciously felt by the patient. This sub-perceptive stimulation approach may be particularly advantageous in clinical settings where stimulation must be administered without the patient or clinical staff being able to detect stimulation, facilitating blinding in clinical trials and enabling continuous treatment without patient distraction or discomfort.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

As used herein, the phrase “primary brain injury” refers to injuries directly resulting from the initial event associated with the brain injury. Primary brain injury can cause the displacement of the physical structure of the brain. As used herein, primary brain injuries include, but are not limited to, intracerebral hemorrhages (ICH), subdural hemorrhages, intraparenchymal hemorrhage (IPH), subarachnoid hemorrhages (SAH), epidural hemorrhages, cerebral contusions, cerebral lacerations, axonal stretch injuries, other traumatic brain injuries, ischemic strokes, etc. As used herein, the phrase “secondary brain injury” refers to brain injuries that arise after (e.g., gradually arise, spontaneously arise, etc.) a primary brain injury due to one or more metabolic processes of a patient. That is, secondary brain injuries are the indirect result of the initial event associated with the brain injury. As used herein, secondary brain injuries include, but are not limited to, edema, ischemia, hypoxia, hypotension, intracranial pressure, brain herniation, etc. Treatment for a brain injury includes directly treating the primary brain injury (e.g., via medication, via surgery, etc.) and taking measuring to prevent and/or mitigate the potentially harmful effects of secondary brain injuries.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

Optional” or “optionally” means that the subsequently described event or circumstance may or may not occur, and that the description includes instances where the event occurs and instances where it does not.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

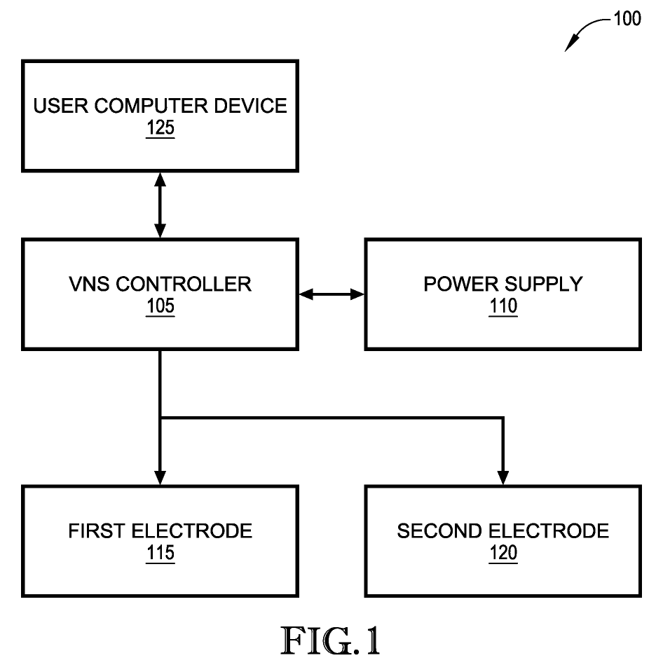

FIG. 1 illustrates a system 100 for providing vagal nerve stimulation to a patient in accordance with at least one embodiment.

The system 100 includes a VNS controller 105. The VNS controller 105 can be a computer device, such as a tablet, laptop, desktop, or other dedicated computer device including at least one processor in communication with at least one memory device. The VNS controller 105 can also include a user interface that that allows the VNS controller 105 to present information to a user and receive user inputs.

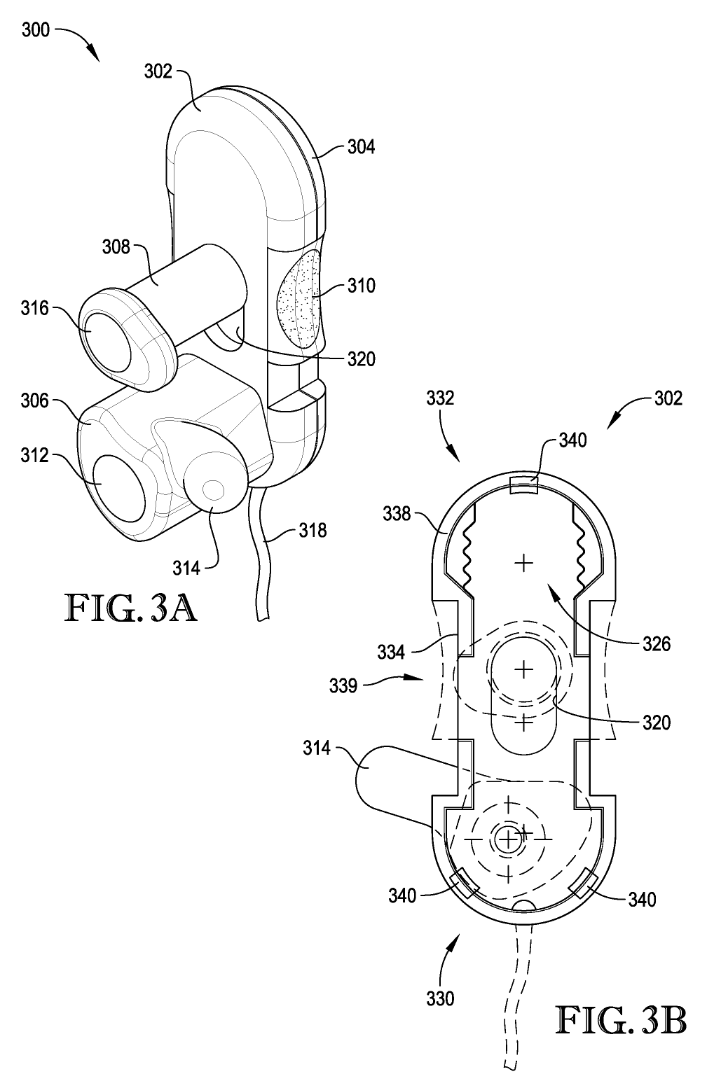

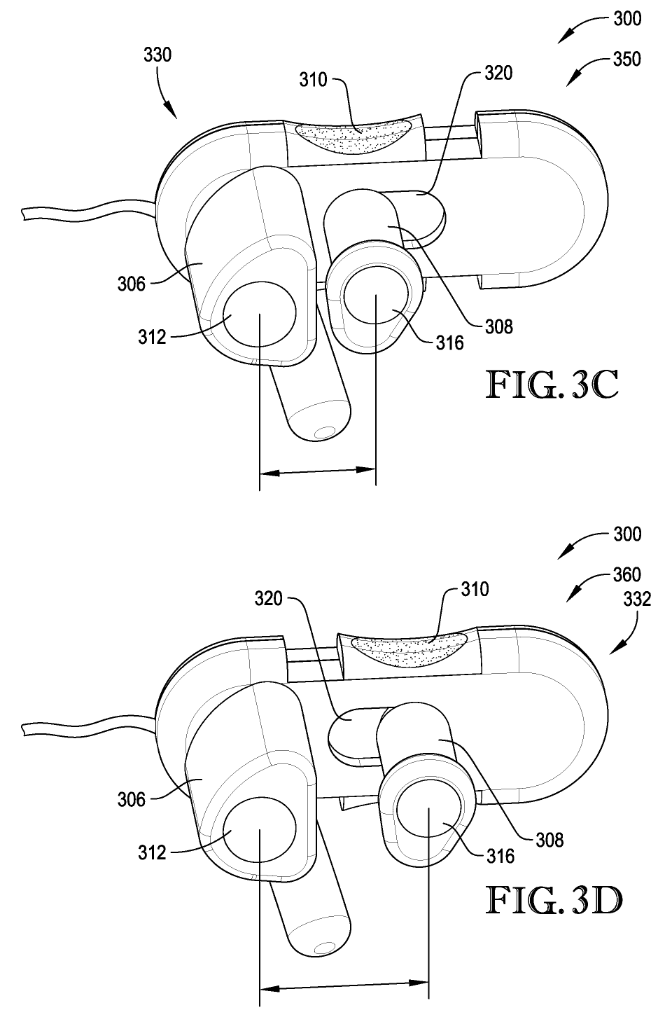

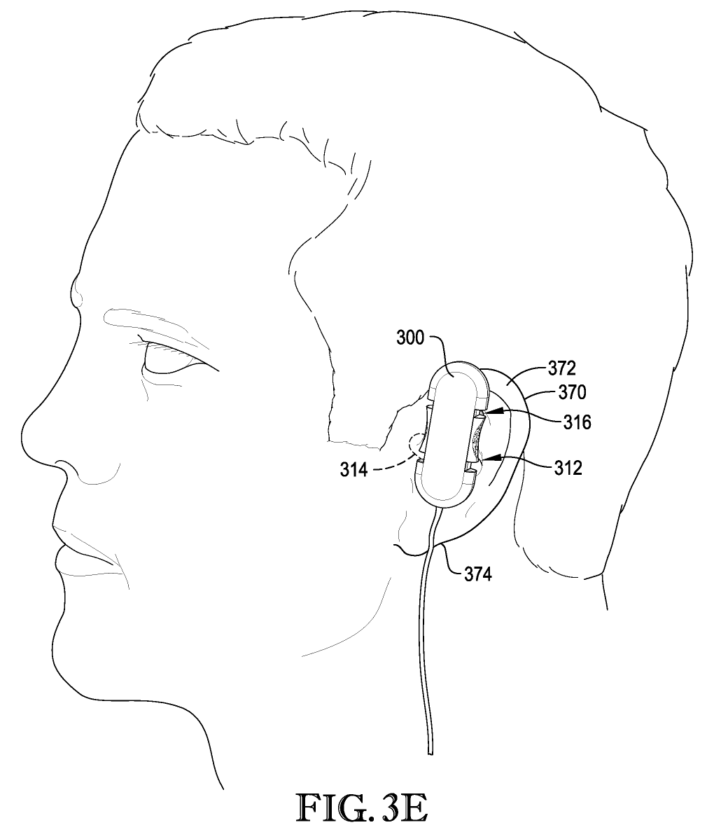

The VNS controller 105 is in communication with a power supply 110 configured to provide electrical stimulation. The VNS controller 105 can also be in communication with one or more electrodes of a neuromodulation device, such as a first electrode 115 and a second electrode 120. The first electrode 115 and the second electrode 120 are configured to provide the electrical stimulation to the patient. In some embodiments, first electrode 115 and second electrode 120 are permanent, re-usable electrodes. In other embodiments, the first electrode 115 and the second electrode 120 are disposable, single use electrodes. In still further embodiments, one or more of the first electrode 115 and the second electrode 120 are implanted in the patient to stimulate the vagus nerve. In additional embodiments, the first electrode 115 and the second electrode are temporarily attached to the patient’s ear to stimulate the vagus nerve. While one example set of electrodes is depicted in FIG. 1 (e.g., the electrodes 115, 120, etc.), In other embodiments, the VNS controller 105 can apply stimulation to a patient’s vagus nerve via a different set of electrodes. An earpiece including electrodes compatible with the VNS controller 105 and implemented in accordance with teachings of this disclosure is described below in conjunction with FIGS. 3A-3E.

In at least one embodiment, the VNS controller 105 is configured to provide treatment to the vagus nerve by electrically stimulation for a period of twenty minutes. In at least one embodiment, the attributes of the electrical stimulation are 20 Hertz (Hz), 250 microseconds (μs), and 0.4 milliamps (mA). In other embodiments, the current can range between 0.4 and 8 mA. The attributes of the electrical stimulation stay the same throughout the treatment. In at least one further embodiment, the electrical stimulation is performed twice a day. In at least one embodiment, the attributes of the electrical stimulation are selected to maximize vagus somatosensory evoked potentials while avoiding perception of pain.

In the exemplary embodiment, the VNS controller 105 controls the output of the power supply 110 to provide the electrical stimulation via the first electrode 115 and the second electrode 120.

In some further embodiments, VNS controller 105 is in communication with one or more user computer devices 125. The user computer device 125 may provide information to the VNS controller 105, such as one or more attributes of the patient that may alter the electrical stimulation applied to the patient. Furthermore, the user computer device 125 may provide timing information to the VNS controller 105, such as when to apply the electrical stimulation. Moreover, the user computer device 125 can receive information from the VNS controller 105, such as what were the attributes of the electrical stimulation that was applied to the patient.

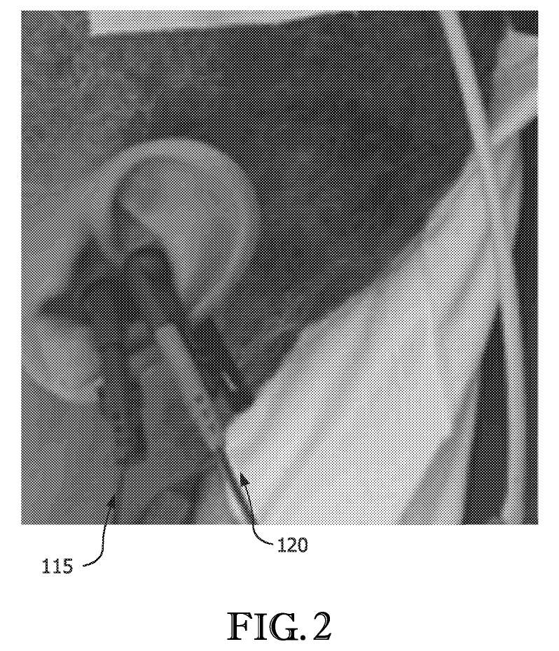

FIG. 2 illustrates placement of the electrodes 115 and 120 (shown in FIG. 1) of a neuromodulation device for non-invasive transcutaneous vagus nerve stimulation using the system 100 (shown in FIG. 1). In FIG. 2, the VNS controller 105 (shown in FIG. 1) is a part of a portable TENS (transcutaneous electrical nerve stimulation) unit. The TENS is connected to two the two electrodes 115 and 120.

In the exemplary embodiment, the first electrode 115 and the second electrode 120 are placed along the concha of the ear to stimulate the vagus nerve where the auricular branch travels in the pinna of the ear. In the exemplary embodiment, the first electrode 115 and the second electrode are attached to the patient’s left ear.

FIG. 3A illustrates a perspective view of an earpiece 300 for non-invasive transcutaneous vagus nerve stimulation using the system 100 of FIG. 1. The earpiece 300 is a neuromodulation device. In the illustrated embodiment of FIG. 3A, the earpiece 300 includes a body 302, a lid 304, a first boss 306, a second boss 308, and a slider 310. In the illustrated embodiment of FIG. 3A, the first boss 306 includes a first electrode 312 and an ear canal interface 314. The second boss 308 includes a second electrode 316 and a slider 310. The earpiece 300 includes an electrical interface 318, which can electrically couple the earpiece 300 to the VNS controller 105 of FIG. 1.

The body 302, which is the main structural feature of the earpiece 300, is an oblong shape member configured to fit in a patient’s ear. The body 302 is described below in additional detail in conjunction with FIG. 3B. With reference still to FIG. 3A, the body 302 includes a slot 320 from which the second boss 308 extends.

The bosses 306, 308 of the earpiece 300 extend from the body 302. As used herein, the first boss 306 and the second boss 308 are also referred to as “extensions,” “protrusions,” and “members.” The bosses 306, 308 support and house the electrodes 312, 316. In the illustrated embodiment of FIG. 3A, the bosses 306, 308 extend from a first side of the body 302 (e.g., the side configured to face the head of a patient, etc.) and the lid 304 is coupled to a second side of the body 302 (e.g., the side configured to face away from the patient, etc.).

The first boss 306 is rigidly fixed relative to the body 302 (e.g., not moveable relative to the body 302, etc.). In other suitable embodiments, the first boss 306 can be moveable relative to the body 302. For example, the distance of the first electrode 312 from the body 302 can be adjustable. In other suitable embodiments, the end of the first boss 306 can be rotatable coupled to the body 302 and the first electrode 312 is offset from the center of the first boss 306. In some such embodiments, rotation of the first boss 306 relative to the body 302 enables the location of the first electrode 312 to account for anatomical variance of the patient’s ear (e.g., account for anatomical differences regarding the relative position of the patient’s cavum and the patient’s ear canal, etc.).

The second boss 308 is rigidly connected to the slider 310. As illustrated in FIG. 3A, the second boss 308 is moveable relative to the body 302 and the first boss 306. Particularly, the distance between the first boss 306 and the second boss 308 can be selectively adjusted by moving the slider 310, thereby adjusting the position of the second boss 308 within the slot 320. In some suitable embodiments, the position of the slider 310 (e.g., the position of the second boss 308 and the second electrode 316, etc.) can be retained via the abutment of the lid 304 and the body 302. In some such embodiments, the position of the slider 310 can be adjusted by separating the lid 304 and the body 302 and articulating slider 310 relative to the body 302. In other embodiments, the position of the slider 310 can be adjusted while the lid 304 is coupled to the body 302. For example, the slider 310 can coupled to the body 302 via an interference fit. When the sides of the slider 310 are manually depressed, the interference fit is released, which enables movement of the slider 310 until the sides of the slider 310 is released, thereby reestablishing the interference fit.

In the illustrated embodiment of FIG. 3A, the first boss 306 includes ear canal interface 314, which extends therefrom. The ear canal interface 314 is configured to be positioned in the ear canal of the patient receiving taVNS. The ear canal interface 314 is also referred to herein as an “ear canal member” and/or an “ear canal boss.” The placement of the ear canal interface 314 facilitates retention of the earpiece 300 on the patient during taVNS. In the illustrated embodiment, the ear canal interface 314 includes a smooth outer diameter. In some suitable embodiments, the outer diameter of the ear canal interface 314 is textured and/or includes an elastomer (e.g., a natural rubber, an artificial rubber, a soft plastic, a foam, etc.) to facilitate the retention of the earpiece to the ear of the patient. In some embodiments, the ear canal interface 314 is absent. In some such embodiments, the earpiece 300 can be retained on the ear of the patient via any suitable mechanism (e.g., an over the ear strap, an around the end strap, a clip on the ear ridge, an adhesive, etc.).

The electrodes 312, 316 are conductive elements configured to abut the concha of the patient during taVNS and conduct an electrical signal thereto. The electrodes 312, 316 are similar to the electrodes 115, 120 of FIG. 1 excepted as noted otherwise. The first electrode 312 is configured to abut the cavum of the patient and the second electrode 316 is configured to abut the cymba of the patient during application of taVNS. In the illustrated embodiment, the first electrode 312 and the second electrode 316 are disposed at the ends of the bosses 306, 308, respectively, distal to the body 302. The electrodes 312, 316 are electrically coupled to the electrical interface 318.

The electrical interface 318 extends from the bottom of the body 302. In other embodiments, the electrical interface 318 can extend from a different portion of the body 302 (e.g., a top of the body 302, a side of the body 302, etc.). The electrical interface 318 includes one or more wires (e.g., insulated wires, etc.). In some embodiments, the electrical interface 318 can be coupled to the VNS controller 105 of FIG. 1.

The earpiece 300 is an adjustable external medical device that facilitates the application of taVNS via the external ear of the patient. In one suitable embodiment. the earpiece 300 is reusable. That is, the earpiece 300 can be used to apply taVNS to multiple patients following cleaning and/or sterilization. The earpiece 300 is adjustable to facilitate anatomical variability of the ears of different patient (e.g., different ear shapes, different ear sizes, etc.). Particularly, people have variable distances between their cymbas and their cavums. As used herein, this distance is referred to as the cymbal-cavum distance of a patient. This adjustability of the earpiece 300 facilitates the quick application of the earpiece 300 to patients at risk of secondary brain injuries and the consistent application of stimulation of the auricular branch of the vagus nerve via the VNS controller 105 across a diverse patient population.

The structural pieces of the earpiece 300 (e.g., the body 302, the lid 304, the bosses 306, 308, the slider 310, etc.) can be composed of any suitable non-conductive rigid material, such as a plastic (e.g., a medical grade plastic, nylon, polypropylene (PP), polyethylene (PE), polyvinyl Chloride (PVC), and polycarbonate (PC), etc.) and/or a ceramic, glass, a natural material (e.g., a fiber, a wood, etc.). The structural pieces of the earpiece 300 can be manufactured via 3-dimensional printing, injection molding, extrusion, thermoforming, extrusion, etc. The electrodes 312, 316 can be composed of any suitable conductive material, such as a metal (e.g., copper, aluminum, gold, platinum, etc.) a conducting polymer, a carbon-based conductor, etc.

FIG. 3B is a front view of the body 302 of the earpiece 300, in which the lid 304 has been removed. The removal of the lid 304 reveals an interior 326 of the body 302. As used herein, the interior 326 defines an enclosure of the earpiece 300 when the lid 304 is coupled to the body 302. In some embodiments, the electrodes 312, 316 are coupled to the electrical interface 318 via one or more electrical connections positioned in the interior 326. In some embodiments, the interior 326 includes one or more sensors (e.g., sensors for measuring patient attributes, sensors for measuring qualities of the electrical signal being applied via the electrodes 312, 316, etc.). In the illustrated embodiment of FIG. 3B, the body is generally pill-shaped (e.g., stadium shaped, etc.). In other suitable embodiments, the body 302 can have any suitable shape (e.g., circular, cylindrical, polygonal, ear-shaped, etc.

With reference still to FIG. 3B, the body 302 includes a first end 330 and a second end 332. The body 302 also includes a neck 334 disposed between the first end 330 and the second end 332. In the illustrated embodiment, the first boss 306 and the first electrode are disposed at the first end 330. When the earpiece 300 is positioned on the patient, the first end 330 is configured to be adjacent to the ear lobe of the patient and the second end 332 is configured to be distal to the ear lobe of the patient. In the illustrated embodiment of FIG. 3B, the body 302 is wider at the ends 330, 332 than the body 302 is in the neck 334. The neck 334 defines a rail on which the slider 310 of FIG. 3A slides along during adjustment of the earpiece 300.

The slot 320 of the body 302 is a through hole disposed in the neck 334. In the illustrated embodiment of FIG. 3B, the slot 320 is equally spaced from the first end 330 and the second end 332. In other suitable embodiments, the slot 320 can be offset from one of the ends 330, 332 (e.g., closer to the first end 330, closer to the second end 332, etc.) In the illustrated embodiment, the slot 320 is pilled-shaped. In other suitable embodiments, the slot 320 can have any suitable shape including rectangular.

As illustrated in FIG. 3B, the body 302 includes a wall 338 extending around the perimeter thereof. The wall 338 partially defines the interior 326 of the body 302. In the illustrated embodiment, the wall 338 includes an opening 339 positioned in the neck 334 and equally spaced between the ends 330, 332. The opening 339 is aligned with the slot 320. When the earpiece 300 is assembled, the opening 339 is coupled to receive the sides of the slider 310 of FIG. 3A. Movement of the slider 310 within the opening 339 facilitates the adjustment of the position of the second boss 308 and thereby facilitates the adjustability of the earpiece 300.

With reference still to FIG. 3B, the body 302 includes tabs 340 disposed at the ends 330, 332. The tabs 340 facilitate the coupling of the lid 304 to the body 302. Particularly, the tabs 340 can receive corresponding clips on the lid 304 to form an interference coupling (e.g., an interference fit, etc.) between the lid 304 and the body 302. In the illustrated embodiment, the body 302 includes 3 tabs (e.g., one tab at the second end 332 and two tabs at the first end 330, etc.). In other suitable embodiments, the body 302 can include a different quantity and/or distribution of the tabs 340.

FIG. 3C illustrates a perspective view of the earpiece 300 in a contracted position 350. As seen in FIG. 3C, the second boss 308 is disposed in the slot 320 such that second boss 308 is adjacent to the first end 330 and the slider 310 is positioned in the opening 339 adjacent to the first end 330. In the contracted position 350 (as seen in FIG. 3C), the earpiece 300 is adjusted for patients who have comparably short cavum-cymba distances. That is, the electrodes 312, 316 are in relatively close position in the contracted position 350.

FIG. 3D illustrates a perspective view of the earpiece in an expanded position 360. In the expanded position, the second boss 308 is disposed in the slot 320 such that second boss 308 is adjacent to the second end 332 and the slider 310 is positioned in the opening 339 adjacent to the second end 332. In the expanded position 360, the earpiece 300 is adjusted for patients who have comparably large cavum-cymba distances. That is, the electrodes 312, 316 are in relative spaced position in the expanded position 360.

FIGS. 3C and 3D illustrate two relative positions of the electrodes 312, 316 of the earpiece 300 (e.g., the contracted position 350 of FIG. 3C, the expanded position 360 of FIG. 3D, etc.). It should be appreciated that the contracted position 350 of FIG. 3C and the expanded position 360 of FIG. 3D are the extrema positions of the electrodes 312, 316 (e.g., a closest distance between the electrodes 312, 316, a farthest distance between the electrodes 312, 316, etc.) and the earpiece 300 can be articulated into a plurality of positions between the contracted position 350 of FIG. 3C and the expanded position 360 of FIG. 3D.

FIG. 3E illustrates a perspective view of the earpiece 300 coupled to the patient’s ear 370 in an application position. In the application position, the earpiece 300 is ready to apply taVNS to the patient (e.g., the electrodes 312, 316 of the earpiece 300 are positioned in the concha of the patient’s ear 370, etc.). In the illustrated embodiment, the body 302 extends vertically between a helix 372 of the ear 370 and an ear lobe 374 of the ear 370. The second end 332 of the earpiece 300 abuts the helix 372 and the ear canal interface 314 is inserted into an ear canal of the patient’s ear 370. In some suitable embodiments, prior to the coupling of the earpiece 300 to the patient’s ear 370, the distance between the cymba and the cavum of the ear 370 can be measured by a healthcare professional. The distance between the electrodes 312, 316 can then be suitably adjusted prior to the coupling of the earpiece 300 to the patient’s ear 370.

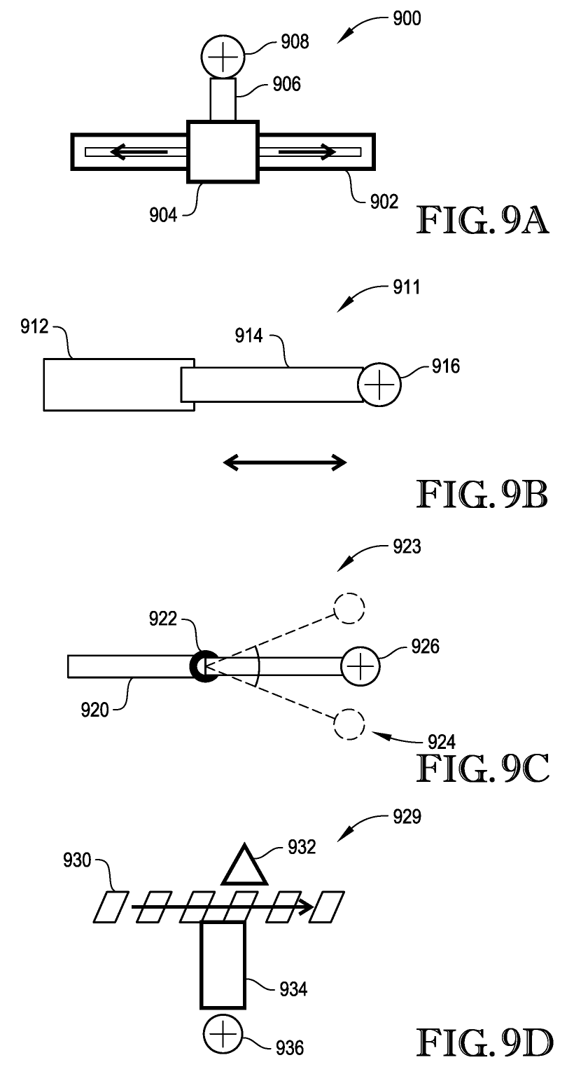

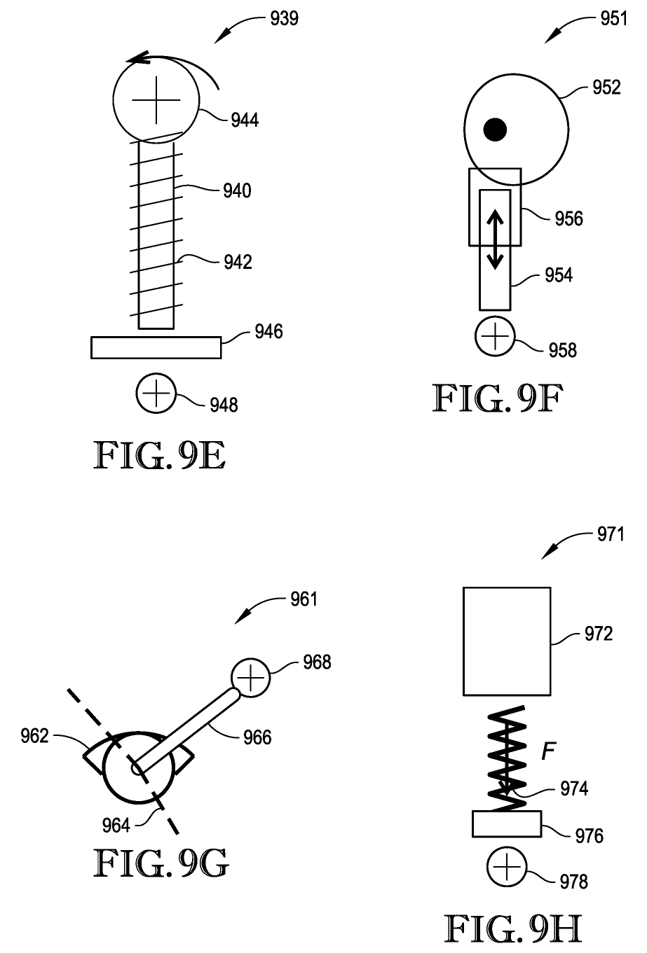

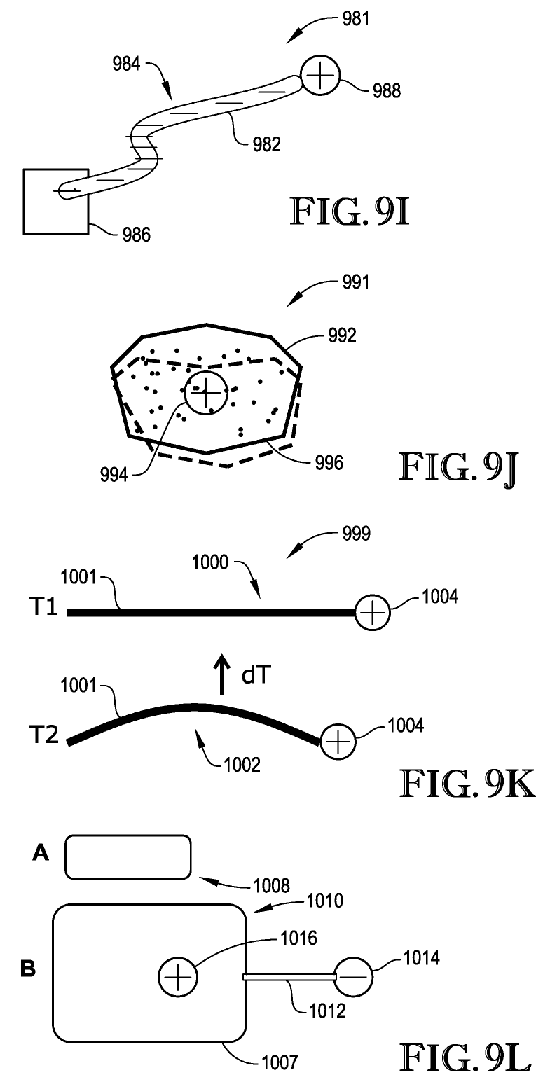

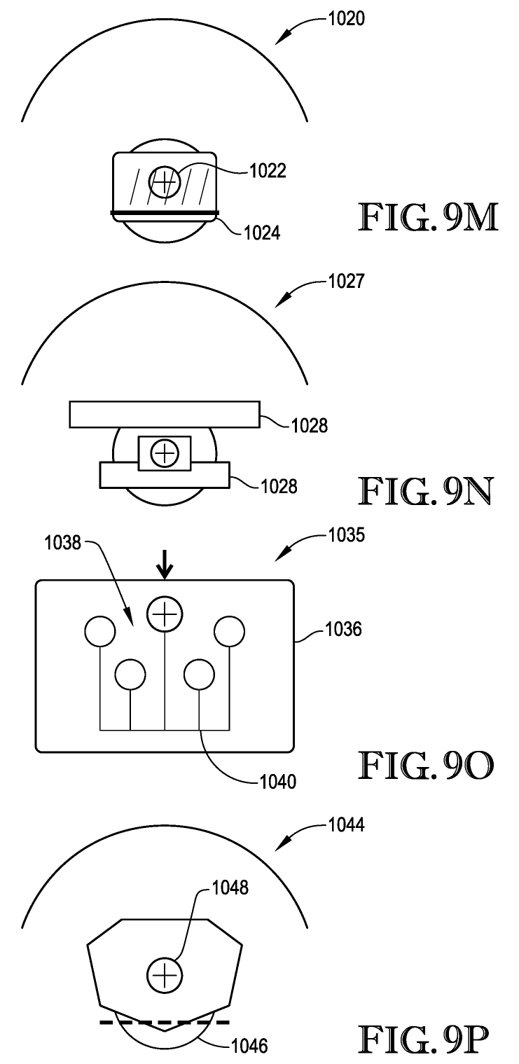

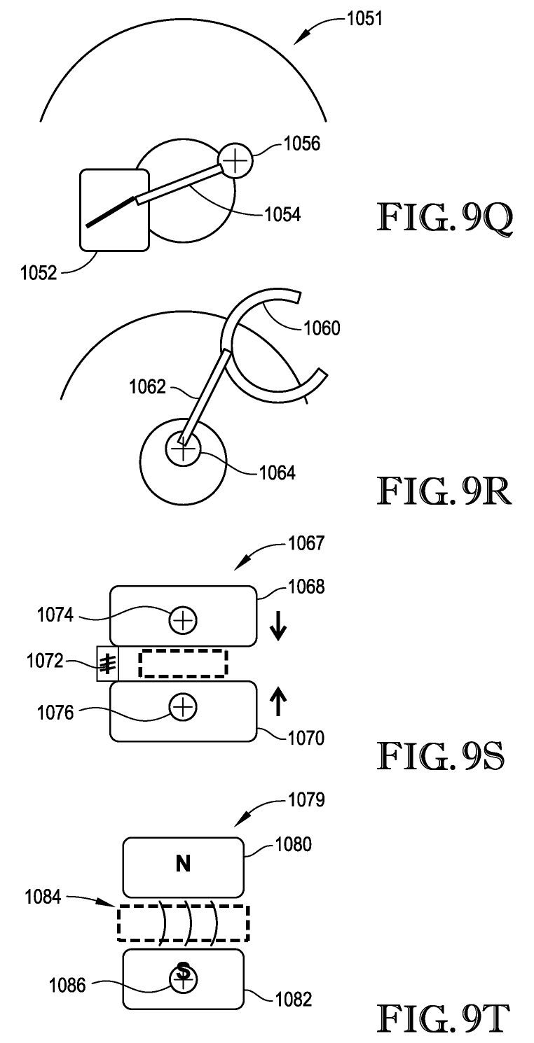

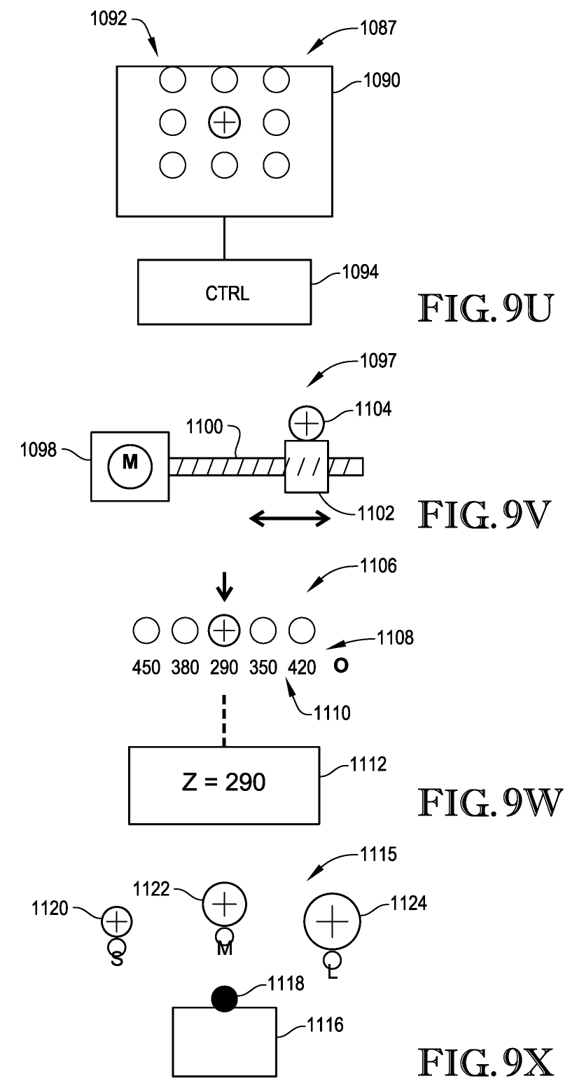

It should be appreciated that other positioning and/or adjustment mechanisms can be used in conjunction with the earpiece 300 of FIGS. 3A-3E. Alternative positioning and/or adjustment mechanisms useable with the earpiece 300 and other neuromodulation devices implemented in accordance with teachings of this disclosure (e.g., a neuromodulation disposed about the neck of a patient, etc.) are described below in conjunction with FIGS. 9A-9X.

### ALTERNATIVE FORM FACTORS FOR VAGAL NERVE STIMULATION

In addition to the wired earpiece configuration described above in conjunction with FIGS. 3A-3E, the systems and methods disclosed herein may be implemented using alternative form factors for delivering vagal nerve stimulation. These alternative form factors include wireless self-contained auricular devices and transvascular stimulation approaches, each offering distinct advantages for particular clinical applications and patient populations.

In some embodiments, the vagal nerve stimulation system comprises a self-contained wireless auricular device configured to be positioned within the ear without external wires or tethered connections. The self-contained wireless device includes an integrated housing that encapsulates the components of the auricular device including one or more electrodes, a power source, control circuitry, and a wireless communication module. The housing is configured to fit securely within the concha region of the patient’s ear, with the electrodes positioned to contact the cymba conchae and/or cavum conchae. The power source comprises one or more rechargeable batteries (e.g., lithium-ion batteries, lithium-polymer batteries, etc.) or primary batteries sized to provide sufficient energy for extended stimulation sessions. In some embodiments, the wireless device includes inductive charging capabilities allowing the device to be recharged without physical connections. The control circuitry includes a microprocessor or microcontroller configured to generate the electrical stimulation signals according to pre-programmed or wirelessly transmitted parameters. The wireless communication module enables bidirectional communication with an external device (e.g., a smartphone, tablet, dedicated controller, etc.) using wireless protocols such as Bluetooth, Bluetooth Low Energy (BLE), Wi-Fi, or near-field communication (NFC). The wireless self-contained device may include onboard memory for storing stimulation protocols, usage data, and patient-specific parameters. In some embodiments, the wireless device includes one or more sensors for monitoring physiological parameters such as heart rate, skin impedance, temperature, or motion, enabling closed-loop adjustment of stimulation parameters based on patient responses.

In other embodiments, VNS may be delivered via a transvascular approach utilizing catheter-based electrode systems positioned within blood vessels adjacent to the vagus nerve. The cervical vagus nerve descends within the carotid sheath, positioned posterior to and between the internal jugular vein and the carotid artery (common carotid artery proximally, internal carotid artery distally). This anatomical relationship enables transvascular access to the vagus nerve via either the internal jugular vein or the carotid artery. In some embodiments, a catheter-based electrode system is advanced through the venous system (e.g., via femoral vein access, subclavian vein access, or jugular vein access) and positioned within the internal jugular vein at a level corresponding to the cervical vagus nerve. The catheter includes one or more electrodes configured to deliver electrical stimulation through the vessel wall to activate the adjacent vagus nerve. In other embodiments, the catheter-based electrode system is positioned within the carotid artery (common carotid or internal carotid) to stimulate the vagus nerve from an arterial approach. The transvascular electrodes may be configured for temporary placement during acute interventions or for longer-term placement with appropriate fixation mechanisms.

The transvascular approach offers several potential advantages including the ability to stimulate the main vagal trunk rather than an auricular branch, potentially enabling more robust activation of vagal efferent pathways. Additionally, the transvascular approach may be particularly suitable for patients who are already undergoing catheter-based procedures (e.g., diagnostic angiography, endovascular treatment of aneurysms, etc.) or for critically ill patients in the intensive care unit who may not be able to cooperate with external device placement. In some embodiments, the transvascular stimulation system includes a stimulation catheter coupled to an external pulse generator via a percutaneous connection. In other embodiments, the transvascular system comprises an implantable pulse generator positioned subcutaneously and connected to the transvascular electrodes via subcutaneous leads. The stimulation parameters for transvascular vagal nerve stimulation may differ from those used for transcutaneous auricular stimulation due to the different electrode-nerve interface characteristics.

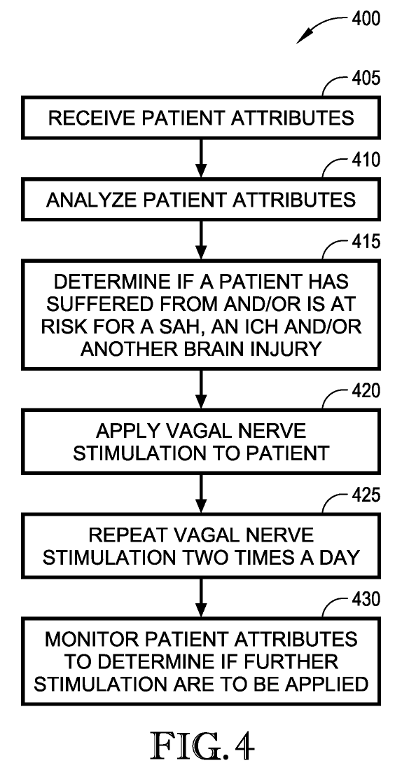

FIG. 4 illustrates a process 400 for providing vagal nerve stimulation using the system 100 (shown in FIG. 1). In the exemplary embodiment, portions of process 400 are performed by a user computer device 125 (shown in FIG. 1), which may be, but is not limited to, a tablet, a laptop, a desktop, and/or and other computer device including at least one processor in communication with at least one memory device. Additionally, portions of process 400 are performed by the VNS controller 105 (shown in FIG. 1).

At block 405, the user computer device 125 receives patient attributes. For example, the patient attributes could be received when the patient checks in or by retrieving the patient history. The patient attributes can include but are not limited to, height, weight, gender, heart rate, blood pressure, medical history, reasons for admittance, bloodwork results (e.g., a presence of NfL, etc.), vital statistics, presence/location of an aneurysm on vascular imaging, Hunt and Hess grade of SAH, Fisher grade of SAH, and other attributes. In some embodiments, the patient attributes can further include CT (Computed tomography) imaging of SAH with a cerebral aneurysm confirmed with a four-vessel cerebral angiogram.

At block 410, the patient attributes are analyzed. In some embodiments, the patient attributes (e.g., test results, demographic risk factors, etc.) can be reviewed by a healthcare professional, the VNS controller 105, and/or an AI system to identify attributes relative to diagnosing a brain injury, identifying a risk for developing a brain injury, and/or identifying a risk for developing a secondary brain injury following a brain injury.

At block 415, it is determined if the patient has suffered from and/or is at risk for a spontaneous subarachnoid hemorrhage (SAH), an intracerebral hemorrhage (ICH), and/or another brain injury based on the analyzed patient attributes. In some embodiments, a healthcare professional can determine if a patient has suffered from and/or is at risk of a secondary brain injury following a primary brain injury. In some embodiments, the patient attributes analyzed during the execution of block 410 can be compared to one or more indicators of a brain injury. In some embodiments, a patient’s bloodwork (e.g., analyzed from a plasma sample, analyzed from a serum sample, etc.) can be reviewed to determine elevated levels of a biomarker, such as neurofilament light chain (NfL), are present. NfL is a structural protein composed of myelinated axons whose presence in blood rise proportionally with neuronal damage, such as the neuronal damage that occur after a brain injury (e.g., ICH, etc.). As such, NfL is a sensitive and specific biomarker of neuroaxonal injury (e.g., ICHs, etc.).

At block 420, if the patient is determined to be at risk for a SAH, ICH, and/or secondary injury following thereof the healthcare provider may apply vagal nerve stimulation to the patient using the system 100 (shown in FIG. 1). In some embodiments, the application of taVNS occurs within 30 minutes of a brain injury (e.g., 30 minutes post-ICH, 30 minutes post SAH, etc.). In some embodiments, the healthcare provider attaches two electrodes (e.g., the first electrode 115 of FIG. 1 and second electrode 120 of FIG. 1, etc.) to the concha of the left ear of the patient. Following the attachment of the electrodes 115, 120, the VNS controller 105 then provides an electrical signal (e.g., a current, etc.) through the electrodes 115 and 120.

In other embodiments, the earpiece 300 of FIGS. 3A-3E can be coupled to the patient’s ear (e.g., the ear 370 of FIG. 3E, etc.). In some embodiments, the geometry of the patient’s ear can be analyzed (e.g., the cavum-cymba distance of the ear, the relative position of the ear canal and the cymba, etc.). In some embodiments, the earpiece 300 can be adjusted to fit a patient’s ear prior to and/or while the earpiece 300 is being coupled thereto.

In one embodiment, the electrical signal has the following attributes: a frequency of 20 Hz, a pulse width of 250 μs, and an amplitude (e.g., an intensity, etc.) 8 mA. In other embodiment, electrical signal can have properties. In some embodiments, the current can range from 0.4 mA to 8 mA, the pulse width can range between 10 microseconds and 600 microseconds, and frequency can range between 1 Hz and 2 kilohertz (kHz). In other embodiments, the frequency can range between 20 Hz and 200 Hz.

In some embodiments, the electrical signal of the stimulation can be applied with a sub-perceptive intensity (e.g., an electrical signal with an intensity that is not felt by the patient, etc.). In some embodiments, the electrical signal can have an amplitude of less than 0.5 milliamps. In some embodiments, application of electrical signals with sub-perceptive intensity has secondary benefits in reducing the metabolic rate of particular regions of the brain. In some embodiments, the application of electrical signals to the vagus nerve via the electrodes 115, 120 of FIG. 1 and/or the electrodes 312, 316 of FIG. 3A can reduce the metabolic rate (e.g., brain activity, etc.) of particular brain regions (e.g., the insula, orbitofrontal cortex, etc.) associated with inflammation. In some embodiments, the reduced metabolic rate in such brain regions reduces the likelihood of secondary brain injury caused by inflammation. In some embodiments, the metabolic rate of a brain regions can be measured by high gamma brain wave activity therein. Accordingly, applications of taVNS (e.g., via the electrodes 115, 120, via the electrodes 312, 316, etc.) in the sub-preceptive range can reduce high gamma brain wave activity in brain regions associated with inflammatory response. In other embodiments, the taVNS applied by the electrodes 115, 120 can be in the preceptive range (e.g., stimulation with an intensity that a patient can feel, etc.) and/or maximum tolerable range (e.g. a stimulation with an intensity just below a patient’s pain tolerance, etc.).

In some embodiments, the taVNS is applied via the electrodes for a period of at least twenty minutes. In some embodiments, the duration of the taVNS is between 1 minutes and 2 hours . After the application of the taVNS, t the stimulation is discontinued and the electrodes 115, 120 are removed from the patient’s ear. In some embodiments, the electrical stimulation remains at the same attributes (e.g., a same frequency, a same intensity, a same pulse width, etc.) during the entire period of stimulation. In other embodiments, the electrical stimulation is started at a lower intensity and the VNS controller 105 increases the intensity over time. Additionally or alternatively, other attributes of the electrical stimulation can be varied over the duration of the electrical stimulation.

In some embodiments, the attributes of the stimulation can change depending on other factors, such as the demographics of the patient (e.g., age, sex, etc.), the location of the primary injury (e.g., an ICH, an SAH, etc.), and/or the severity of the primary injury (e.g., a bleed rate of the injury, a volume of blood in the injury, etc.). In some embodiments, the demographic information of the patient, the location of the primary injury, and/or the severity of the injury can be used to determine which ear the taVNS is applied to (e.g., the left ear, the right ear, etc.), the intensity of the stimulation, the pulse width of the stimulation, the frequency of the stimulation, the taVNS duration, etc.).

At block 425, the healthcare provider and the VNS controller 105 repeats the vagal nerve stimulation at least twice a day. In some embodiments, after a first stimulation, the electrodes 115, 120 can be recoupled to a patient’s ear within 24 hours (e.g., after 6 hours, after 12 hours, etc.) of the first stimulation and a second stimulation can be applied via the electrodes 115, 120.

At block 430, after the electrical stimulation is complete, the patient’s vital statistics (e.g., pulse rate, blood pressure, blood oxygen level, cognitive markers, etc.) can be monitored to determine if further stimulations are to be applied. In some embodiments, a patient can be continuously physiologically monitored and samples (e.g., blood samples, cerebrospinal fluid samples, etc.) can be extracted to measure biomarkers (e.g., NfL, IL-6, etc.) associated with systemic and/or local inflammatory responses. Additionally or alternatively, collect brain images (e.g., via magnetic resonance imaging (MRI), via computerized tomography (CT) scanning, via positron emission tomography (PET), via single photon emission computed tomography (SPECT), via diffusion tensor imaging (DTI), via a brain ultrasound, etc.) can be reviewed to identify development of inflammatory and/or edema in a patient. If further stimulation is needed, the process 400 can be repeated.

In the exemplary embodiment, the VNS controller 105 stimulates a patient's vagus nerve with an electrical signal to achieve a therapeutic effect for treating the inflammation, where the inflammation is related to a brain injury, such as subarachnoid hemorrhage (SAH) and/or an intracerebral hemorrhage (ICH). In some embodiments, the electrical signal includes a signal intensity (e.g., a current, an amplitude in a range 0.4 mA between 8 mA, a pulse width in a range 10μs-600μs, a signal frequency in range of 20 Hz – 2 kHz, and a signal on-time of at least twenty minutes (e.g., thirty minutes, 1 hour, 2 hours, etc.). In some embodiments, the VNS controller 105 also re-stimulates the patient’s vagus nerve with a second stimulation within 24 hours of a first stimulation. In some embodiments, the properties of the electrical signal for the first stimulation and a second stimulation are the same (e.g., a same duration, a same intensity, a same pulse width, a same frequency, etc.). In other embodiments, the properties of the first stimulation and the second stimulation are different (e.g., a different duration, a different intensity, a different pulse width, a different frequency, etc.). In some embodiments, the properties of the second stimulation applied by the VNS controller 105 can be varied based on updated health parameters of the patient. In some embodiments, the properties of the first stimulation and the second stimulation remain constant for the duration of stimulation(s).

In the exemplary embodiment, the stimulation is the transcutaneous stimulation of the vagus nerve, wherein the stimulation is provided via the first electrode 115 and the second electrode 120. In some embodiments, the first electrode 115 and the second electrode 120 are attached to the concha and cymba, respectively, of the patient’s left ear. In other embodiments, the first electrode 115 and the second electrode 120 can be applied to a patient’s right ear.

The stimulation is provided to the auricular branch of the vagus nerve where the vagus nerve travels in the pinna of the ear. In some further embodiments, the stimulation is paired with an antibiotic, an anti-inflammatory medication, and/or medication based on the health parameters of the patient.

In addition to providing electrical stimulation to the patient, the healthcare provider also monitors multiple vital signs of the patient. In some embodiments, the patient’s plasma and cerebrospinal fluid (CSF) are collected periodically, such as every three days, to quantify inflammatory markers. The rates of cerebral vasospasm and chronic hydrocephalus can be assessed. In addition, functional outcomes via modified Rankin Scale (mRS) scores can be collected.

In some embodiments, blood and Cerebrospinal fluid (CSF) samples are collected prior to the first electrical stimulation of the patient. The samples can be processed to provide the complete blood count with differential and CSF cell count with differential. For evaluation of the cytokines, the samples are centrifuged, aliquoted, and stored in a deep freezer until ready for processing.

Frozen supernatant plasma and CSF are slowly thawed and then analyzed in duplicate with multiplex kits (Thermofisher Scientific, Waltham, MA) for multiple pro-inflammatory cytokines: IL-1β, IL-2, IL-5, IL-6, IL-8, IL-12, IL-13, IL-17, TNF-α, GM-CSF, and IFN-γ; and anti-inflammatory cytokines: IL-4 and IL-10. The concentration of each antigen is calculated by plotting the expected concentration of the standards against the multiplex fluorescent immunoassay generated by each standard. A 4-parameter logistic regression is then used for the best-fit curve. Protein concentration is reported as pg/mL.

One goal is quantified continuous measures of the serum and CSF markers of inflammation (i.e., IL-6, TNF-α, etc.) collected at two time points, baseline (before treatment) and day 13 after treatment. The taVNS impact on inflammatory markers can then be examined via a linear mixed model, where time (i.e., 0- and 13-days post-treatment), treatment (i.e., taVNS vs. Sham), and time-treatment interaction are the fixed effects, and the dependency of measurements clustered within each individual patient are accounted for.

In addition to analyze how the taVNS alters the development of the secondary SAH sequela of radiographic vasospasm, and its mediation by the inflammatory response, initial diagnostic imaging, patients will undergo a repeat computed tomography or catheter angiogram seven days after admission. Additionally, further vascular imaging will be performed if there is clinical concern per the intensive care or neurosurgical teams for clinical vasospasm or stroke. For both planned and indicated imaging sessions, each vascular imaging study is reviewed to describe the imaging as it relates to vasospasm as none, mild (< 25% stenosis), moderate (25%– 50% stenosis), or severe (> 50% stenosis) narrowing of at least one major intracranial artery, as previously described. Additional clinical metrics related to vasospasm can also be used. Specifically, these can include the following: 1) blood pressure augmentation while in the intensive care unit, 2) number of vascular imaging sessions, 3) treatments performed during catheter angiogram (e.g., intraarterial vasodilators), 4) use of intrathecal vasodilators, and 5) CT imaging identified strokes and parenchymal volume of strokes.

The above analysis is to fully quantify the incidence, severity, and treatment response to radiographic vasospasm in SAH patients. The goal of the electrical stimulation treatment is to reduce radiographic vasospasm, as well as the need for vasospasm-related interventions like blood pressure augmentation, angioplasty, or intraarterial/intrathecal medications to negate spasm. The reduction of these findings can be associated with lowered CSF inflammatory makers. The taVNS can be correlated with lower blood pressure goals and reduced number of vasospasm interventions. These altered radiographic and clinical changes can also be correlated with a concomitant reduction in CSF inflammatory cytokines.

In a further embodiment, another goal includes defining how taVNS alters key clinical metrics associated with CSF malabsorption after SAH. taVNS can lead to a reduction in duration of EVD drainage and rate of ventricular shunting for chronic hydrocephalus. In this embodiment, specific details of a patient’s clinical course as it relates to impaired CSF absorption and hydrocephalus is defined with primary outcome metrics including 1) need for surgical placement of permanent CSF diversion such as a ventriculoperitoneal or ventriculoarterial shunt prior to discharge from the hospital and 2) duration of external ventricular drainage.

The goal is to have SAH patients and ICH patients treated with taVNS to display a significant reduction in duration of EVD placement and lowered rates of chronic hydrocephalus requiring ventricular shunt. Further, these improvements correlate with reduced CSF inflammatory markers. This effect may be more pronounced in higher grade hemorrhage where the incidence of hydrocephalus is higher.

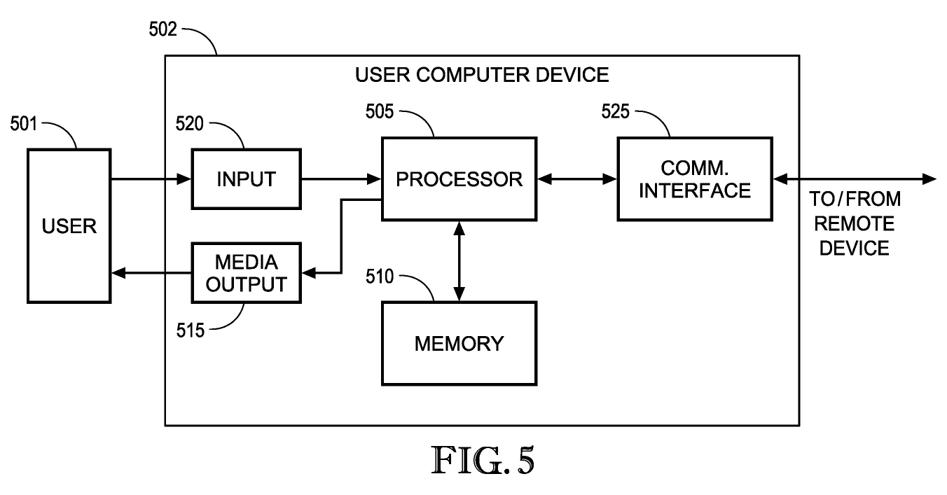

FIG. 5 illustrates an example configuration of a client system shown in FIG. 1, in accordance with one embodiment of the present disclosure. User computer device 502 is operated by a user 501. User computer device 502 may include, but is not limited to, VNS controller 105 and user computer device 125 (both shown in FIG. 1). User computer device 502 includes a processor 505 for executing instructions. In some embodiments, executable instructions are stored in a memory area 510. Processor 505 may include one or more processing units (e.g., in a multi-core configuration). Memory area 510 is any device allowing information such as executable instructions and/or transaction data to be stored and retrieved. Memory area 510 may include one or more computer-readable media.

User computer device 502 also includes at least one media output component 515 for presenting information to user 501. Media output component 515 is any component capable of conveying information to user 501. In some embodiments, media output component 515 includes an output adapter (not shown) such as a video adapter and/or an audio adapter. An output adapter is operatively coupled to processor 505 and operatively couplable to an output device such as a display device (e.g., a cathode ray tube (CRT), liquid crystal display (LCD), light emitting diode (LED) display, or “electronic ink” display) or an audio output device (e.g., a speaker or headphones). In some embodiments, media output component 515 is configured to present a graphical user interface (e.g., a web browser and/or a client application) to user 501. A graphical user interface may include, for example, patient attributes or the attributes of the electrical stimulation. In some embodiments, user computer device 502 includes an input device 520 for receiving input from user 501. User 501 may use the input device 520 to, without limitation, select to apply the electrical stimulation to the patient. Input device 520 may include, for example, a keyboard, a pointing device, a mouse, a stylus, a touch sensitive panel (e.g., a touch pad or a touch screen), a gyroscope, an accelerometer, a position detector, a biometric input device, and/or an audio input device. A single component such as a touch screen may function as both an output device of media output component 515 and input device 520.

User computer device 502 may also include a communication interface 525, communicatively coupled to a remote device such as a VNS controller 105 or a user computer device 125. Communication interface 525 may include, for example, a wired or wireless network adapter and/or a wireless data transceiver for use with a mobile telecommunications network.

Stored in memory area 510 are, for example, computer-readable instructions for providing a user interface to user 501 via media output component 515 and, optionally, receiving and processing input from input device 520. The user interface may include, among other possibilities, a web browser and/or a client application. Web browsers enable users, such as user 501, to display and interact with media and other information typically embedded on a web page or a website provided by a server. A client application allows user 501 to interact with, for example, VNS controller 105. For example, instructions may be stored by a cloud service and the output of the execution of the instructions sent to the media output component 515.

### IMAGING EVIDENCE OF EDEMA REDUCTION WITH taVNS

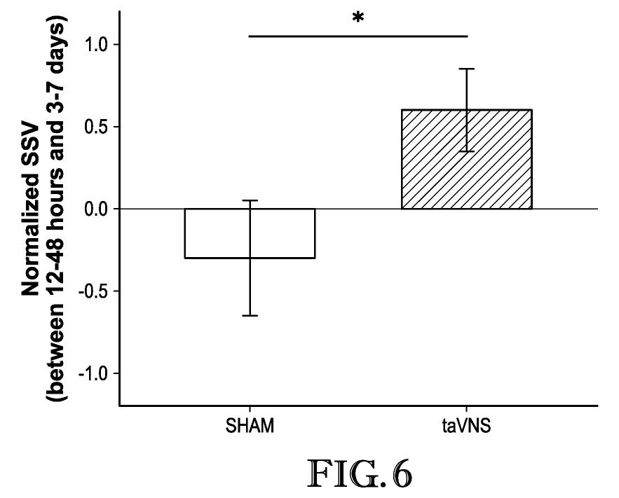

In addition to inflammatory biomarker assessment, the inventors conducted imaging studies to quantify the effect of taVNS on cerebral edema in patients with brain hemorrhages. With reference to FIG. 6, in a cohort of twenty-seven SAH patients, an artificial intelligence (AI) algorithm was applied to computed tomography (CT) imaging to measure Selective Sulcal Volume (SSV), a validated surrogate marker of early brain injury. The SSV represents the amount of cerebrospinal fluid (CSF) within the sulci above the ventricles, and reduced SSV is a sensitive, quantitative biomarker of global cerebral edema. Patients who received taVNS treatment demonstrated an increase in SSV over time, indicating improvement in cerebral edema, while patients receiving sham treatment did not demonstrate such improvement. Specifically, the difference in normalized SSV from 12-48 hours to 3-7 days post-hemorrhage was significantly more positive in patients receiving taVNS treatment compared to sham (t-statistic = -2.7, p-value = 0.036, N(sham) = 3, N(taVNS) = 5). These findings demonstrate that taVNS can have a meaningful impact on global cerebral edema in the setting of intracranial hemorrhage.

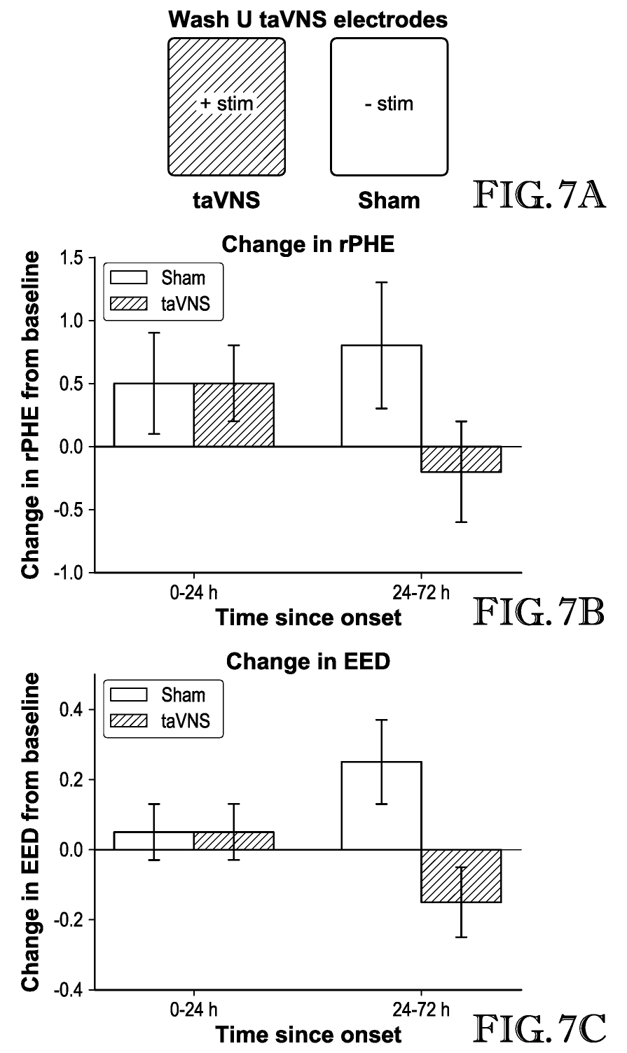

With reference to FIGS. 7A-7C, a pilot study was performed in ten patients presenting with intraparenchymal hemorrhage (IPH), a type of ICH. Patients were randomized to receive either taVNS treatment or sham stimulation. The treatment protocol consisted of twice-daily sessions of 20 minutes duration, with stimulation parameters of 20 Hz frequency, 250 microsecond pulse width, and 0.5 mA amplitude. Sham stimulation consisted of identical electrode placement without electrical current delivery. The taVNS electrodes were custom-designed to specifically stimulate the concha region of the auricle (FIG. 7A), which is known to be predominantly innervated by the auricular branch of the vagus nerve. Importantly, this electrode design enables optimal blinding because the stimulation is sub-perception, preventing both patients and care staff from determining treatment assignment.

CT scans obtained on admission and throughout the ICU course were analyzed using a previously validated AI pipeline to measure hematoma volume and perihematomal edema (PHE). Two quantitative metrics of PHE were computed: relative perihematomal edema (rPHE) and edema extension distance (EED). Baseline-corrected values were calculated at the subject level relative to the initial scan, and measurements were grouped into two time windows based on last known normal: 0-24 hours and 24-72 hours. The effect of taVNS was quantified by computing effect sizes (Cliff’s δ) comparing treatment groups across all available scans and subjects within each time window.

With reference to FIG. 7B, baseline-corrected rPHE was lower in the taVNS treatment group compared to the sham group after 24 hours, with minimal difference during the first 24 hours (Cliff’s δ = 0.02) but a substantial treatment effect in the 24-72 hour window (Cliff’s δ = -0.70). With reference to FIG. 7C, baseline-corrected EED demonstrated a similar pattern, with no difference during the first 24 hours (Cliff’s δ = 0.00) and a large treatment effect in the 24-72 hour window (Cliff’s δ = -0.70). These preliminary data demonstrate the feasibility of the AI-based imaging analysis approach and provide support that taVNS can mitigate perihematomal edema in the setting of ICH. The temporal pattern of the treatment effect, becoming apparent in the 24-72 hour window, is consistent with the expected time course of the neuroinflammatory response and blood-brain barrier disruption following ICH.

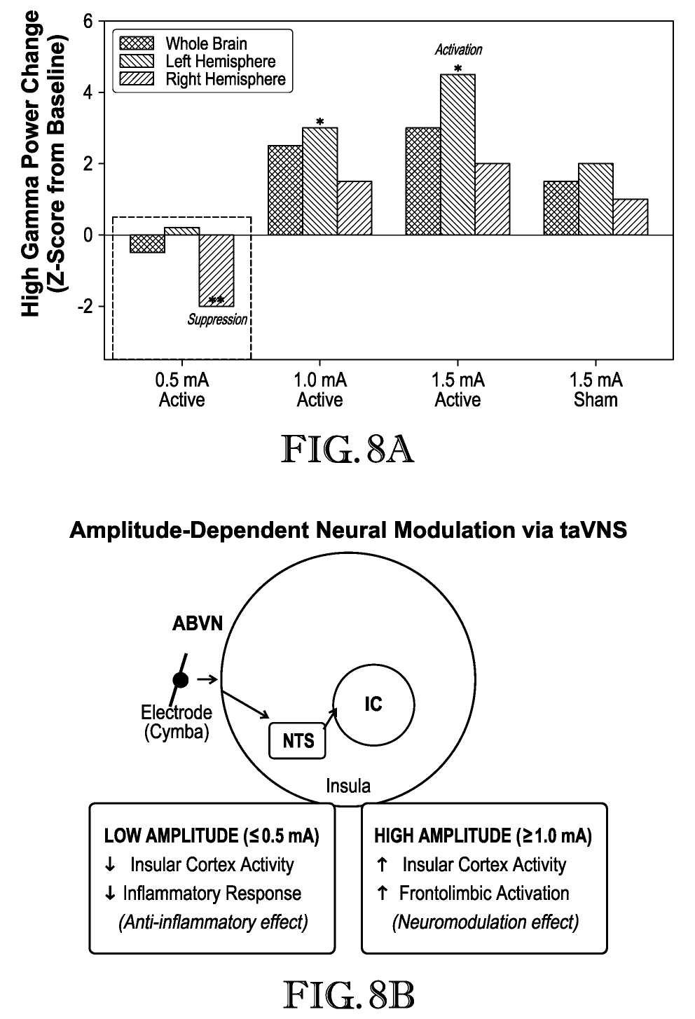

With reference to FIG. 8, direct neurophysiological evidence of the central effects of taVNS was obtained through intracranial recordings in human subjects. In a study using stereotactic electroencephalography (sEEG), brain activity was recorded from patients with intractable epilepsy (n = 8 included for analysis) while they underwent both active and sham taVNS. Electrodes were positioned to record from frontolimbic-insular regions of interest, including the insular cortex, orbitofrontal cortex (OFC), anterior cingulate cortex (ACC), inferior frontal gyrus (IFG), and middle frontal gyrus (MFG). Both active and sham stimulation conditions were administered at 30 Hz frequency, 250 µs pulse width, with 30 second trains separated by 1 second inter-train intervals, for a total stimulation duration of one minute. Active taVNS amplitudes included 0.5, 1.0, and 1.5 mA, while sham taVNS was delivered to the earlobe at 1.5 mA. High gamma power between 95-170 Hz was quantified as the primary indicator of local cortical activity.

With reference to FIG. 8A, high gamma activity was found to be amplitude-dependent, with different taVNS amplitudes producing distinct and qualitatively different effects on cortical activity. Active taVNS at 1.0 and 1.5 mA amplitudes resulted in significantly increased high gamma activity across the brain, with more prominent responses observed in the left hemisphere and in the insular cortex and orbitofrontal cortex. Importantly, low-amplitude stimulation at 0.5 mA produced the opposite effect: suppression of high gamma activity, particularly in the right hemisphere (p = 0.018). The right insular cortex was among the brain regions showing decreased high gamma activity during 0.5 mA active taVNS.

With reference to FIG. 8B, these findings have important implications for the therapeutic application of taVNS in treating brain swelling and inflammation. Given the insular cortex’s established role as a key hub for immunoception and immune regulation, as described hereinabove, reduced cortical activation produced by low-amplitude stimulation (0.5 mA and below) may contribute to the therapeutic anti-inflammatory effects observed in clinical trials. Stimulation at low amplitude may attenuate insular cortex activity that encodes and maintains peripheral inflammatory states, thereby interrupting the bidirectional amplification loop between brain and immune system that can perpetuate neuroinflammation. This mechanism provides a neurophysiological basis for the observation that sub-perceptive stimulation amplitudes (e.g., 0.5 mA and below) can produce clinically meaningful reductions in cerebral edema and inflammatory markers in patients with brain hemorrhage.

Referring now to FIGS. 9A-9X, FIGS. 9A-9X illustrates various electrode positioning and adjustability mechanisms for accommodating anatomical variations between patients and ensuring optimal electrode contact with auricular target regions. In some examples, the positioning and adjustability mechanisms of FIGS. 9A-9X can be used in conjunction with the adjustable earpiece 300 of FIGS. 3A-3E and/or another stimulation device. For example, the some or all of the electrode positioning and adjustability mechanisms of FIGS. 9A-9X can be used in a neuromodulation device that is couplable to a neck of a patient (e.g., around a neck of a patient, etc.). FIGS. 9A-9H depict mechanical adjustment systems. FIGS. 9I-9L depict conformable adjustment systems. FIGS. 9M-9P depict adhesive adjustment systems. FIGS. 9Q-9T depict fixation and retention mechanisms. FIGS. 9U-9X depict fixation and retention mechanisms.

FIG. 9A is a schematic illustration of a first electrode positioning system including a sliding rail mechanism 900 wherein an electrode 908 is mounted on a slider 904 that travels linearly along a rail 902. In FIG. 9A, the electrode 908 is coupled to the slider 904 via an arm 906.

FIG. 9B is a schematic illustration of a second electrode positioning system including a telescoping arm mechanism 911. In FIG. 9B, the second electrode positioning system includes an outer tube 912 and an inner tube 914 providing adjustable extension length for positioning electrode 916.

FIG. 9C is a schematic illustration of a third electrode positioning system including a hinged pivot joint 923. In FIG. 9C, the third electrode positioning system includes a rotating arm 924 pivots about pivot point 922 to enable angular positioning of electrode 926.

FIG. 9D is a schematic illustration of a fourth electrode positioning system including a ratchet mechanism 929. In FIG. 9D, the fourth electrode positioning system includes ratchet teeth 930 and pawl 932 for incremental positioning with positive locking of electrode arm 934.

FIG. 9E is a schematic illustration of a fifth electrode positioning system including a threaded screw mechanism 939. In FIG. 9E, the fifth electrode positioning system includes a screw body 940 having threads 942, an adjustable knob 944, a platform 946, and an electrode 948. In one aspect, rotation of the adjustment knob 944 drives the screw body 940 to provide fine linear adjustment of the platform 946 and the electrode 948 along the threads 942.

FIG. 9F is a schematic illustration of a sixth electrode positioning system including a cam mechanism 951. In FIG. 9E, the sixth electrode positioning system includes an eccentric cam 952, a follower 954, a guide housing 956, and an electrode 958. In some aspects, rotation of the eccentric cam 952 drives the follower 954 within the guide housing 956 to position the electrode 958.

FIG. 9G is a schematic illustration of a seventh electrode positioning system including a ball and socket joint 961. In FIG. 9G, the seventh electrode positioning system includes a socket 962 (e.g., a socket housing 962, etc.), a ball 964, an arm 966, and an electrode 968. In some aspects, manual rotation of the ball 964 within the socket enables multi-axis articulation of the arm 966. Movement of the arm 966 enables the location of the electrode 968 to be adjusted.

FIG. 9H is a schematic illustration of an eighth electrode positioning system including a spring-loaded mechanism 971. In FIG. 9H, the eighth electrode positioning system includes a housing 972, a spring 974, a plunger 976, and an electrode 978. In some aspects, the force applied by the spring 974 within the housing 972 biases plunger 976 and electrode 978 to provide self-adjusting contact pressure against the skin of the patient (e.g., the auricular surface, etc.).

FIG. 9I is a schematic illustration of a nineth electrode positioning system including a flexible gooseneck arm 981. The nineth electrode positioning system is a conformable material-based mechanism. In FIG. 9H, the nineth electrode positioning system includes a segmented flexible arm 982 with segments 984, a base 986, and an electrode 988. The segments 984 extend from the base 986 to the electrode 988. In some examples, each of the segments 984 is hingedly coupled to adjacent ones of the segments 984. Additionally or alternatively, the segments 984 are composed of a flexible, articulate material that maintains its shape after manual adjustment (e.g., a plastic, an elastomer, etc.). Manual adjustment of the nineth electrode positioning system enables the electrode 988 to be positioned by a user of a device including the nineth electrode positioning system.

FIG. 9J is a schematic illustration of a tenth electrode positioning system including a conformable viscoelastic pad 991. In FIG. 9J, the tenth electrode positioning system includes a flexible pad 992 and the embedded electrode 944. In some embodiments, the flexible pad 992 is composed of foam. In other embodiments, the flexible pad is composed of another viscoelastic material (e.g., rubber, plastic, etc.). The flexible pad 992 can be conformed into a conformed shape 996, such that the flexible pad 992 conforms to the shape of a patient’s ear and the embedded electrode 994 is positioned for VNS (e.g., taVNS, etc.).

FIG. 9K is a schematic illustration of an eleventh electrode positioning system including a shape memory alloy (SMA) adjustment mechanism 999 include an SMA member 1001 and an electrode 1004. In some embodiments, the SMA member 1001 is a wire (e.g., a copper-aluminum-nickel alloy, a nickel titanium alloy, etc.). In FIG. 9K, the SMA member 1001 of the eleventh electrode positioning system is configurable between a first configuration 1000 at temperature T1 and a second configuration 1002 at temperature T2. It should be appreciated that the SMA member 1001 is position able into a plurality of different positions based on the temperature applied to the SMA member 1001, which enables the positioning of the electrode for VNS (e.g., taVNS, etc.).

FIG. 9L is a schematic illustration of a twelfth electrode positioning system including an inflatable bladder 1007, an air tube 1012, a pump 1014, and an electrode 1016. In FIG. 9L, the inflatable bladder 1007 is adjustable between a deflated state 1008 and an inflated state 1010. In some embodiments, the inflatable bladder 1007 can inflated and conformed to the patient (e.g., conformed to auricular contours of a patient, etc.). The inflation of the inflatable bladder 1007 positions the electrode 1016 for VNS (e.g., taVNS, etc.).

FIG. 9M is a schematic illustration of a thirteenth electrode positioning system including a conductive hydrogel adhesive layer 1020. In FIG. 9M, the thirteenth electrode positioning system includes an electrode 1022 and a skin contact surface 1024. In some aspects, the conductive hydrogel adhesive layer 1020 provides both adhesion and electrode contact between the skin of the patient and the electrode 1022.

FIG. 9N is a schematic illustration of a fourteenth electrode positioning system including an adhesive tape strip system 1027. In FIG. 9N, the fourteenth electrode positioning system includes medical adhesive tape strips 1028, an electrode unit 1030, and an electrode 1032. The electrode unit 1030 is a housing that contains the electrode 1032. The medical adhesive tape strips 1028 are medical grade adhesives that enable the securing the electrode unit 1030 to the skin of a patient (e.g., an auricular surface of the patient, etc.).

FIG. 9O is a schematic illustration of a fifteenth electrode positioning system including a selectable adhesive electrode array 1035. In FIG. 9O, the fifteenth electrode positioning system includes a flexible substrate 1036, a plurality of electrodes 1038, and a plurality of traces 1040. In one aspect, the plurality of electrodes 1038 are connected by the traces 1040. After coupling the flexible substrate 1036 to a patient (e.g., an ear of a patient, etc.). The properly positioned electrode can be selected (e.g., via a mechanical switch, via a graphical user interface, etc.) to facilitate stimulation of the vagus nerve.

FIG. 9P is a schematic illustration of a sixteenth electrode positioning system including a biocompatible silicone adhesive patch 1044. In FIG. 9P, the biocompatible silicone adhesive patch 1044 includes an adhesive layer 1046 and electrode 1048. In one aspect, the adhesive layer 1046 can be coupled to the skin of a patient (e.g., an ear of the patient, a skin of the patient, which facilitates stimulation of the vagus nerve of the patient via the electrode 1048.

FIG. 9Q is a schematic illustration of a seventeenth electrode positioning system including an ear canal anchor 1051. In FIG. 9Q, the seventeenth electrode positioning system includes an ear canal insert 1052, an arm 1054, and an electrode 1056. The ear canal insert 1052 can be inserted into the ear canal of a patient, which provides retention of the electrode 1056 on the ear of the patient. In FIG. 9Q, the arm 1054 extends to position the electrode 1056 in the concha of the patient.

FIG. 9R is a schematic illustration of a eighteenth electrode positioning system including an over-ear hook 1060, an extension arm 1062, and an electrode 1064. In FIG. 9R, the over-ear hook 1060 can engage with the helix of a patient’s ear (e.g., a ridge of the ear, a top of the ear, a bottom of the ear, etc.). The arm 1062 extends from the over-ear hook 1060 to position the electrode 1064 in the concha of the patient.

FIG. 9S is a schematic illustration of a nineteenth electrode positioning system including a concha clip 1067. In FIG. 9S, the concha clip 1067 includes an upper jaw 1068, lower jaw 1070, a spring hinge 1072, a first electrode 1074, and a second electrode 1076. In one aspect, the electrodes 1074, 1076 are positioned in the jaws 1068, 1070, respectively. The spring hinge 1072 bias the jaws 1068, 1070. The concha clip 1067 can be manually expanded and clamped about the ear tissue of the patient, such that the electrodes 1074, 1076 are properly positioned in the concha of the patient’s ear.

FIG. 9T is a schematic illustration of a twentieth electrode positioning system including a magnetic attachment system 1079. In FIG. 9T, the magnetic attachment system 1079 includes an inner magnet 1080, an outer magnet 1082, and an electrode 1086. The magnets 1080, 1082 generate a magnetic attraction force 1084, which biases the magnets 1080, 1082 together. The magnetic attachment system 1079 can be manually expanded and clamped about the ear tissue of the patient, such that the electrodes 1086 is properly positioned in the concha of the patient’s ear.

FIG. 9U is a schematic illustration of a twenty-first electrode positioning system including an electronically switchable electrode array 1087. In FIG. 9U, the electronically switchable electrode array 1087 includes a substrate 1090, an electrode array 1092, and a controller 1094. The substrate 1090 can be positioned in the ear of the patient (e.g., via one of the other positioning systems of FIGS. 9A-9X, etc.) and the active electrode(s) of the electrode array 1092 can be selected by a user via the controller 1094. Selection of the proper electrode facilitates stimulation of the vagus nerve by the electronically switchable electrode array 1087.

FIG. 9V is a schematic illustration of a twenty-second electrode positioning system including a motorized positioning mechanism 1097. In FIG. 9V, the motorized positioning mechanism 1097 includes a motor 1098, a lead screw 1100, a carriage 1102, and an electrode 1104. The motor 1098 can be implemented by an suitable type of electrical motor and/or manually operated motor. In other embodiments, the lead screw 1100 can be implemented by any other type of linear actuator. The electrode 1104 is coupled to the carriage 1102 (e.g., supported by the carriage 1102, housing in the carriage, etc.). During operation, the motor 1098 drives the lead screw 1100 to translate carriage 1102 and electrode 1104, thereby facilitating the positioning of the electrode in the concha of the patient’s ear.

FIG. 9W is a schematic illustration of a twenty-third electrode positioning system including an impedance-guided positioning system 1106 that includes an electrode array 1108, an impedance sensor 1110, and a user interface 1112. In some examples, the user interface 1112 is a graphical display. In other examples, the user interface 1112 can be implemented by any other suitable type of display. The impedance sensor 1110 enables a user of the impedance-guided positioning system 1106 to determine which of the electrodes of the electrode array 1108 should be active based on feedback from the impedance sensor 1110 provided by the user interface 1112.

FIG. 9X is a schematic illustration of a twenty-fourth electrode positioning system including a modular interchangeable electrode system 1115. In FIG. 9X, the modular interchangeable electrode system 1115 includes a base unit 1116, a connector 118, and a plurality of interchangeable electrode tips 1120, 1122, 1124. The plurality of interchangeable electrode tips 1120, 1122, 1124 are electrodes of different sizes (e.g., different lengths, different geometries, different diameters, etc.). The connector 1118 is compatible with (e.g., able to accept, etc.) each of the plurality of interchangeable electrode tips 1120, 1122, 1124. A user of the modular interchangeable electrode system 1115 can select one of the plurality of interchangeable electrode tips 1120, 1122, 1124 based on the anatomy of the patient’s ear, such that the selected one of the plurality of interchangeable electrode tips 1120, 1122, 1124 are properly positioned on the concha of the patient’s ear.

In various embodiments, the electrode positioning system may comprise any combination of the foregoing mechanical, conformable, adhesive, fixation, and electronic mechanisms of FIGS. 9A-9X to accommodate patient anatomical variations and ensure optimal electrode contact with target auricular regions innervated by the auricular branch of the vagus nerve.

A computer program of one embodiment is embodied on a computer-readable medium. In one embodiment, the system is executed on a single computer system, without requiring a connection to a server computer. In another embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Cisco Systems, Inc. located in San Jose, CA). In yet a further embodiment, the system is run on a Mac OS® environment (Mac OS is a registered trademark of Apple Inc. located in Cupertino, CA). In still yet a further embodiment, the system is run on Android® OS (Android is a registered trademark of Google, Inc. of Mountain View, CA). In another embodiment, the system is run on Linux® OS (Linux is a registered trademark of Linus Torvalds of Boston, MA). The application is flexible and designed to run in various different environments without compromising any major functionality. In some embodiments, the system includes multiple components distributed among a plurality of computing devices. One or more components are in the form of computer-executable instructions embodied in a computer-readable medium. The systems and processes are not limited to the specific embodiments described herein. In addition, components of each system and each process can be practiced independently and separately from other components and processes described herein. Each component and process can also be used in combination with other assembly packages and processes.

As used herein, the terms “processor” and “computer” and related terms, e.g., “processing device”, “computing device”, and “controller” are not limited to just those integrated circuits referred to in the art as a computer, but broadly refers to a microcontroller, a microcomputer, a programmable logic controller (PLC), an application specific integrated circuit (ASIC), and other programmable circuits, and these terms are used interchangeably herein. In the embodiments described herein, memory may include, but is not limited to, a computer-readable medium, such as a random-access memory (RAM), and a computer-readable non-volatile medium, such as flash memory. Alternatively, a floppy disk, a compact disc – read only memory (CD-ROM), a magneto-optical disk (MOD), and/or a digital versatile disc (DVD) may also be used. Also, in the embodiments described herein, additional input channels may be, but are not limited to, computer peripherals associated with an operator interface such as a mouse and a keyboard. Alternatively, other computer peripherals may also be used that may include, for example, but not be limited to, a scanner. Furthermore, in the exemplary embodiment, additional output channels may include, but not be limited to, an operator interface monitor.

Further, as used herein, the terms “software” and “firmware” are interchangeable and include any computer program storage in memory for execution by personal computers, workstations, clients, servers, and respective processing elements thereof.

As used herein, the term “non-transitory computer-readable media” is intended to be representative of any tangible computer-based device implemented in any method or technology for short-term and long-term storage of information, such as, computer-readable instructions, data structures, program modules and sub-modules, or other data in any device. Therefore, the methods described herein may be encoded as executable instructions embodied in a tangible, non-transitory, computer readable medium, including, without limitation, a storage device, and a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

Furthermore, as used herein, the term “real-time” refers to at least one of the time of occurrence of the associated events, the time of measurement and collection of predetermined data, the time for a computing device (e.g., a processor) to process the data, and the time of a system response to the events and the environment. In the embodiments described herein, these activities and events may be considered to occur substantially instantaneously.

The computer-implemented methods and processes described herein may include additional, fewer, or alternate actions, including those discussed elsewhere herein. The present systems and methods may be implemented using one or more local or remote processors, transceivers, and/or sensors (such as processors, transceivers, and/or sensors mounted on vehicles, stations, nodes, or mobile devices, or associated with smart infrastructures and/or remote servers), and/or through implementation of computer-executable instructions stored on non-transitory computer-readable media or medium. Unless described herein to the contrary, the various steps of the several processes may be performed in a different order, or simultaneously in some instances.

Additionally, the computer systems discussed herein may include additional, fewer, or alternative elements and respective functionalities, including those discussed elsewhere herein, which themselves may include or be implemented according to computer-executable instructions stored on non-transitory computer-readable media or medium.

In the exemplary embodiment, a processing element may be instructed to execute one or more of the processes and subprocesses described above by providing the processing element with computer-executable instructions to perform such steps/sub-steps, and store collected data (e.g., trust stores, authentication information, etc.) in a memory or storage associated therewith. This stored information may be used by the respective processing elements to make the determinations necessary to perform other relevant processing steps, as described above.

The embodiments described herein may be implemented as part of one or more computer components, such as a client device, system, and/or components thereof, for example. Furthermore, one or more of the embodiments described herein may be implemented as part of a computer network architecture and/or a cognitive computing architecture that facilitates communications between various other devices and/or components. Thus, the embodiments described herein address and solve issues of a technical nature that are necessarily rooted in computer technology.

Although specific features of various embodiments may be shown in some drawings and not in others, this is for convenience only. In accordance with the principles of the systems and methods described herein, any feature of a drawing may be referenced or claimed in combination with any feature of any other drawing.

Some embodiments involve the use of one or more electronic or computing devices. Such devices typically include a processor, processing device, or controller, such as a general purpose central processing unit (CPU), a graphics processing unit (GPU), a microcontroller, a reduced instruction set computer (RISC) processor, an application specific integrated circuit (ASIC), a programmable logic circuit (PLC), a programmable logic unit (PLU), a field programmable gate array (FPGA), a digital signal processing (DSP) device, and/or any other circuit or processing device capable of executing the functions described herein. The methods described herein may be encoded as executable instructions embodied in a computer readable medium, including, without limitation, a storage device and/or a memory device. Such instructions, when executed by a processing device, cause the processing device to perform at least a portion of the methods described herein. The above embodiments are exemplary only, and thus are not intended to limit in any way the definition and/or meaning of the term processor and processing device.

The computer-implemented methods discussed herein may include additional, less, or alternate actions, including those discussed elsewhere herein. The methods may be implemented via one or more local or remote processors, transceivers, servers, and/or sensors, and/or via computer-executable instructions stored on non-transitory computer-readable media or medium.

Additionally, the computer systems discussed herein may include additional, less, or alternate functionality, including that discussed elsewhere herein. The computer systems discussed herein may include or be implemented via computer-executable instructions stored on non-transitory computer-readable media or medium.

This written description uses embodiments to disclose the invention, including the best mode, and also to enable any person skilled in the art to practice the invention, including making and using any devices or systems and performing any incorporated methods. The patentable scope of the invention is defined by the claims, and may include other embodiments that occur to those skilled in the art. Such other embodiments are intended to be within the scope of the claims if they have structural elements that do not differ from the literal language of the claims, or if they include equivalent structural elements with insubstantial differences from the literal language of the claims.

What is claimed is:

1. A method of reducing cerebral edema in a patient, the method comprising:
identifying the patient as having at least one of 1) having a condition associated with cerebral edema, or 2) being at risk for cerebral edema complications;
positioning, on a skin of the patient, a neuromodulation device having an electrode;
adjusting the neuromodulation device to position the electrode adjacent to at least one of a vagus nerve of the patient or a branch of the vagus nerve; and
emitting an electrical signal at a predetermined intensity to stimulate the vagus nerve of the patient and to reduce cerebral edema by modulating cortical brain activity, thereby reducing metabolic burden on the brain, and activating anti-inflammatory pathways.

2. The method of claim 1, wherein the electrical signal is a first electrical signal and further including stimulating the vagus nerve with a second electrical signal within 24 hours of the emitting of the first electrical signal.

3. The method of claim 2, wherein stimulating the vagus nerve with the second electrical signal includes:
positioning the adjustable earpiece in an application position; and
applying the second electrical signal with same attributes as the first electrical signal.

4. The method of claim 1, wherein the electrical signal has an intensity in a sub-preceptive range.

5. The method of claim 4, wherein the electrical signal has the intensity of less than 0.5 milliamps and the electrical signal has a pulse width between 10 microseconds and 600 microseconds.

6. The method of claim 1, wherein the branch is an auricular branch of the vagus nerve.

7. The method of claim 1, wherein the electrical signal has a frequency between 1 Hertz and 2 kilohertz.

8. The method of claim 1, wherein the condition includes at least one an intracranial hemorrhage, a traumatic brain injury, an ischemic stroke, or a brain tumor.

9. The method of claim 8, wherein the positioning the neuromodulation device includes positioning the neuromodulation device in a concha of the patient.

10. The method of claim 1, further including determining an attribute of the electrical signal based on the at least one (1) demographic information of the patient, or (2) a location of the brain hemorrhage.

11. A system for treating a patient, the system comprising:
an adjustable earpiece including:
a body;
a first boss extending from the body, the first boss including a first electrode; and
a second boss extending from the body, the second boss including a second electrode, the second boss moveable relative to the first boss; and
an electrical stimulation device configured to stimulate a vagus nerve of the patient by applying an electrical stimulation via at least one of the first electrode or the second electrode, the electrical stimulation device electrically coupled to the adjustable earpiece.

12. The system of claim 11, wherein the first electrode is configured to abut a cymba of the patient and the second electrode is configured to abut a cavum of the patient.

13. The system of claim 11, wherein the electrical stimulation has an intensity in a sub-preceptive range.

14. The system of claim 11, wherein the electrical stimulation has an amplitude between 0.2 milliamps and 8 milliamps.

15. The system of claim 11, wherein the electrical stimulation has a pulse width between 10 microseconds and 600 microseconds.

16. The system of claim 11, wherein the electrical stimulation has a frequency between 1 Hertz and 2 kilohertz.

17. The system of claim 11, wherein the electrical stimulation is applied for at least 15 minutes.

18. The system of claim 11, wherein the adjustable earpiece has a first end, a second end, and a rail extending between the first end and the second end, the first electrode being positioned at the first end and the second electrode being moveable along the rail.

19. An earpiece configured to apply an electrical signal to the vagus nerve of a patient, the earpiece comprising:
a body having a first side and a second side;
a lid coupled to the first side of the body;
a first boss extending from the second side of the body, the first boss including a first electrode; and
a second boss extending from the second side of the body, the second boss including a second electrode, the second boss being moveable relative to the first boss; and
a power interface to electrical couple the first electrode and the second electrode to an electrical signal source.

20. The earpiece of claim 19, further including an ear canal member configured to extend into an ear canal of the patient.
